(19) United States
(12) Patent Application Publication
(10) Pub. No.: US 2024/0043544 A1
(43) Pub. Date: Feb. 8, 2024

(54) ANTI-EGFR CHIMERIC ANTIGEN RECEPTORS

(71) Applicant: Seattle Children's Hospital (dba Seattle Children's Research Institute), Seattle, WA (US)

(72) Inventors: Michael C. Jensen, Seattle, WA (US); Jia Wei, Seattle, WA (US)

(21) Appl. No.: 18/265,405

(22) PCT Filed: Dec. 3, 2021
(86) PCT No.: PCT/US2021/061746

§ 371 Date: Jun. 5, 2023

Related U.S. Application Data
(60) Provisional application No. 63/122,839, filed on Dec. 8, 2020.
(60) Provisional application No. 63/234,090, filed on Aug. 17, 2021.

(51) Int. Cl. C07K 16/28

(52) U.S. Cl. CPC C07K 16/2863

(57) ABSTRACT

Disclosed herein is a polynucleotide comprising a human codon-optimized sequence encoding a polypeptide comprising EGFR806CAR. The codon-optimized sequence may be incorporated into a construct comprising an optimal-functioning promoter, spacer, intracellular signaling domain, transmembrane domain, selection marker, at least one self-cleaving peptides, and EFGRt, in order to optimize expression. This sequence may then be expressed in cells, such as T cells, for the treatment or inhibition of a cancer, such as glioblastoma, liquid tumors, or solid tumors.

### CROSS-REFERENCE TO RELATED APPLICATIONS

This application claims priority to U.S. Prov. App. No. 63/234,090 filed Aug. 17, 2021 entitled “ANTI-EGFR CHIMERIC ANTIGEN RECEPTORS”; and U.S. Prov. App. No. 63/122,839 filed Dec. 8, 2020 entitled “CODON-OPTIMIZED EGFR806CAR THERAPEUTIC SEQUENCE,” which are each expressly incorporated by reference herein in its entirety.

### REFERENCE TO SEQUENCE LISTING

The present application is being filed along with a Sequence Listing in electronic format. The Sequence Listing is provided as a file entitled SCRI348WOSEQLIST, created Dec. 1, 2021, which is approximately 11 kilobytes in size. The information in the electronic format of the Sequence Listing is expressly incorporated herein by reference in its entirety.

### FIELD OF THE INVENTION

Aspects of the present disclosure generally relate to anti-EGFR chimeric antigen receptors (CAR), and T cells containing such CARs. Some embodiments pertain to the enhanced expression of anti-EGFR CARs, processes for expressing anti-EGFR CARs, and methods of using the enhanced expression to target cancers such as glioblastoma, liquid tumors, or solid tumors.

### BACKGROUND OF THE INVENTION

Many therapies have been utilized for the treatment of cancer. Today, popular therapies include surgery, chemotherapy, radiation therapy, hormone therapy, targeted drug therapy, and cellular therapy. The cellular therapy for patients suffering from cancer or disease is the injection of cellular material, such as living cells, into a patient in need. This can include the infusion of polyclonal or antigen specific T-cells, activated killer cells, natural killer cells, dendritic cells, or macrophages. Recent advancements have been made in the development of chimeric antigen receptor (CAR) bearing T-cells, a promising therapeutic route for cancer immunotherapy and viral therapy.

CAR T-cell therapy is an immunotherapy, wherein T-cells are isolated in a laboratory and genetically manipulated to express a synthetic receptor, which recognizes a particular antigen or protein displayed on a cell, such as a cancer cell, and then the cells are reinfused into a patient. Clinical trials have shown promising evidence of anti-tumor activity; however, CAR T-cell therapy still suffers from the insufficient activation of cells, short half-lives, and inefficient targeting to cancer tissue. Accordingly, additional approaches to CAR T cell therapy are direly needed.

### SUMMARY OF THE INVENTION

Various embodiments provided herein concern polynucleotides comprising a human codon-optimized sequence encoding an anti-EGFR chimeric antigen receptor (CAR). In some embodiments, said human codon-optimized sequence comprises the sequence set forth in SEQ ID NO: 1. In some embodiments, said polynucleotide further comprises an operably linked promoter. In some embodiments, said promoter comprises an EF1a sequence, or an EF1a/HTLV sequence; preferably a human EF1a sequence, or a human EF1a/HTLV sequence. In some embodiments, said promoter comprises the sequence set forth in SEQ ID NO: 2.

In some embodiments, said polynucleotide further comprises at least one sequence encoding a self-cleavage peptide or an IRES, preferably wherein said sequence encoding said self-cleavage peptide or said IRES is codon-optimized for expression in humans. In some embodiments, said self-cleavage peptide is a 2A self-cleaving peptide, such as P2A or T2A or both. In some embodiments, said sequence encoding the self-cleavage peptide comprises the sequences set forth in SEQ ID NO: 3 and SEQ ID NO: 4.

In some embodiments, said polynucleotide further comprises a sequence encoding one or more selection markers, wherein said sequence encoding said one or more selection markers is preferably codon-optimized for expression in humans. In some embodiments, said one or more selection markers comprises DHFRdm. In some embodiments, said one or more selection markers comprises the sequence set forth in SEQ ID NO: 5.

In some embodiments, said polynucleotide further comprises a sequence encoding EGFRt, preferably wherein said sequence encoding EGFRt is codon optimized for expression in humans. In some embodiments, said sequence encoding said EGFRt comprises the sequence set forth in SEQ ID NO: 6.

In some embodiments, said polynucleotide further comprises a sequence encoding one or more intracellular signaling domains, preferably wherein said sequence encoding said one or more intracellular signaling domains is codon-optimized for expression in humans. In some embodiments, said intracellular signaling domains comprise 41BB, or CD3ξ or both. In some embodiments, said sequence encoding said one or more intracellular signaling domains comprises SEQ ID NO: 7.

In some embodiments, said polynucleotide further comprises a sequence encoding a transmembrane domain, preferably wherein said sequence encoding said transmembrane domain is codon-optimized for expression in humans. In some embodiments, said transmembrane domain comprises CD28tm. In some embodiments, said sequence encoding said transmembrane domain comprises the sequence set forth in SEQ ID NO: 8.

In some embodiments, said polynucleotide further comprises a sequence encoding a spacer, preferably wherein said sequence encoding said spacer is codon-optimized for expression in humans. In some embodiments, said spacer comprises a portion of IgG4, such as a hinge region of IgG4. In some embodiments, said sequence encoding a spacer is set forth in SEQ ID NO: 9. In some embodiments, the sequence of the polynucleotide is set forth in SEQ ID NO: 10.

As further disclosed herein, various embodiments provide an isolated cell comprising any one of the polynucleotides set forth in the above embodiments. In some embodiments, said cell is an immune cell. In some embodiments, said cell is a precursor T cell, or a hematopoietic stem cell. In some embodiments, said cell is a T cell, a B cell, a natural killer cell, an antigen presenting cell, a dendritic cell, a macrophage, or a granulocyte such as a basophil, an eosinophil, a neutrophil, or a mast cell. In some embodiments, said cell is a CD4+ T cell or a CD8+ T cell. In some embodiments, said cell is a CD8+ cytotoxic T cell selected from the group consisting of a naïve CD8+ T cell, a CD8+ memory T cell, a central memory CD8+ T cell, a regulatory CD8+ T cell, an IPS derived CD8+ T cell, an effector memory CD8+ T cell, and a bulk CD8+ T cell. In some embodiments, said cell is a CD4+T helper cell selected from the group consisting of a naïve CD4+ T cell, a CD4+ memory T cell, a central memory CD4+ T cell, a regulatory CD4+ T cell, an IPS derived CD4+ T cell, an effector memory CD4+ T cell, and a bulk CD4+ T cell. In some embodiments, said cell is allogenic to a subject, or is autologous to a subject. In some embodiments, said cell is ex vivo. In some embodiments, said cell is in vivo. In some embodiments, said cell is mammalian. In some embodiments, said cell is human.

As further disclosed herein, various embodiments provide an isolated cell comprising any one of the polynucleotides disclosed herein, and wherein said isolated cell further expresses an antibody or binding fragment thereof or scFv specific for a B cell specific cell surface molecule, such as CD19, CD20, CD1d, CD5, CD19, CD20, CD21, CD22, CD23/Fc epsilon RII, CD24, CD25/IL-2 R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, Clq R1/CD93, CD84/SLAMF5, BAFF R TNFRSFI3C, B220/CD45R, B7-1/CD80, B7-2/CD86, TNFSF7, TNFRSF5, ENPP-1, HVEM/TNFRSF14, BLIMP1/PRDM1, CXCR4, DEP-1/CD148, or EMMPRIN/CD147. In some embodiments, said cell is an immune cell. In some embodiments, said cell is a precursor T cell, or a hematopoietic stem cell. In some embodiments, said cell is a T cell, a B cell, a natural killer cell, an antigen presenting cell, a dendritic cell, a macrophage, or a granulocyte such as a basophil, an eosinophil, a neutrophil, or a mast cell. In some embodiments, said cell is a CD4+ T cell or a CD8+ T cell. In some embodiments, said cell is a CD4+T helper cell selected from the group consisting of a naïve CD4+ T cell, a CD4+ memory T cell, a central memory CD4+ T cell, a regulatory CD4+ T cell, an IPS derived CD4+ T cell, an effector memory CD4+ T cell, and a bulk CD4+ T cell. In some embodiments, said cell is allogenic to a subject, or is autologous to a subject. In some embodiments, said cell is allogenic to a subject, or is autologous to a subject. In some embodiments, said cell is ex vivo. In some embodiments, said cell is in vivo. In some embodiments, said cell is mammalian. In some embodiments, said cell is human.

As further disclosed herein, various embodiments provide a method of inhibiting or treating a cancer in a subject in need thereof, preferably a human, comprising administering any one of the polynucleotides disclosed herein or the cells disclosed herein to said subject. In some embodiments, said administration is conducted by intracranial injection. In some embodiments, said cancer is glioblastoma. In some embodiments, said cancer is a solid tumor. In some embodiments, said solid tumor is selected from the group consisting of a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer.

As further disclosed herein, various embodiments provide a use of any of the polynucleotides disclosed herein or the cells disclosed herein as a medicament. In some embodiments, said polynucleotides of any of the above embodiments or said cells of any of the above embodiments are used for the treatment of a cancer, such as glioblastoma, a leukemia, a lymphoma, a hematological tumor, a liquid tumor, or a solid tumor.

As further disclosed herein, various embodiments provide a method of inhibiting, ameliorating, or treating a cancer in a subject in need thereof, preferably a human, comprising administering any one of the polynucleotides described herein or the cells described herein to said subject in combination with an effective amount of at least one additional anti-cancer agent to provide a combination therapy having an enhanced therapeutic effect. In some embodiments, the cancer is glioblastoma. In some embodiments, the cancer is a solid tumor. In some embodiments, the cancer is a lymphoma, a hematological tumor, or a liquid tumor. In some embodiments, the anti-cancer agent is delivered along with a pharmaceutically acceptable carrier, diluent, excipient or combination thereof.

### BRIEF DESCRIPTION OF THE DRAWINGS

FIG. 1A depicts the schematic of an EGFR806CAR construct sequence controlled by a short promoter (henceforth referred to in drawings as “Short Promoter,” or “SP”).

FIG. 1B depicts the schematic of an EGFR806CAR construct sequence controlled by a long promoter (henceforth referred to in drawings as “Long Promoter,” or “.LP”).

FIG. 1C depicts the schematic of a human codon optimized EGFR806CAR construct sequence controlled by a long promoter (henceforth referred to in drawings as “Codon Optimized,” “CO”, or SEQ ID NO:10).

FIG. 2 depicts the methodology timeline used in primary human T cell studies disclosed herein.

FIG. 3 depicts flow cytometry analysis. T cells were stained with anti-EGFR-Biotin and Streptavidin-APC, six days post transduction and four days post MTX selection. Mock T cells were used as a negative control.

FIG. 4A depicts flow cytometry analysis. T cells were stained with anti-EGFR-Biotin and Streptavidin-APC, eleven days post transduction. Mock T cells were used as a negative control.

FIG. 4B depicts flow cytometry analysis. T cells were stained with EGFRvIII-his and anti-his-APC, eleven days post transduction. Mock T cells were used as a negative control.

FIG. 4C depicts flow cytometry analysis. T cells were stained with Protein L-Biotin and Streptavidin-BV405, eleven days post transduction. Mock T cells were used as a negative control.

FIG. 5A depicts flow cytometry analysis. T cells were stained with anti-EGFR-Biotin and Streptavidin-APC, seven days post rapid expansion. Mock T cells were used as a negative control.

FIG. 5B depicts flow cytometry analysis. T cells were stained with Protein L-Biotin and Streptavidin-BV405, seven days post rapid expansion.

FIG. 5C depicts FACS analysis for levels of staining of cells using an EGFRVIII antigen.

FIG. 5D depicts a bar graph of median fluorescence intensity (MFI) quantification of the PE dye in CD8+ cells shown in FIG. 5C.

FIG. 5E depicts FACS analysis for levels of staining using Erbitux which binds an EGFRt marker co-expressed with each CAR.

FIG. 5F depicts a bar graph MFI quantification of the PE dye in CD8+ cells shown in FIG. 5E.

FIG. 6A depicts a western blot analysis with an anti-CD3 zeta antibody. The molecular weight of the CAR is about 50 kD and the endogenous zeta is about 15 kD. Mock T cells were used as a negative control, and H9 cells expressing 806CAR were used a positive control.

FIG. 6B depicts a western blot analysis with an anti-CD3 zeta antibody.

FIG. 6C depicts a bar graph for quantification of CAR CD3 zeta band levels shown in FIG. 6B.

FIG. 6D depicts a bar graph for gene copy number for the constructs in the cells measured using droplet digital (dd) PCR.

FIG. 6E depicts a bar graph for normalized CD3 zeta intensity from FIG. 6C and normalized again by the average copy number per cell. Cells containing the long promoter construct (HIV7.3).

FIG. 7A depicts a cytokine release assay (BioPlex) for IL2. K562 parental line and K562/OKT3 were used as negative and positive controls, respectively. K562/EGFRvIII line is a target line of 806CAR T cells.

FIG. 7B depicts a cytokine release assay (BioPlex) for TNFs. K562 parental line and K562/OKT3 were used as negative and positive controls, respectively.

FIG. 7C depicts a cytokine release assay (BioPlex) for IFNg. K562 parental line and K562/OKT3 were used as negative and positive controls, respectively.

FIG. 7D depicts a cytokine release assay (MSD for IL2, TNFa, and IFNg in different human donor cells. K562 parental line and K562/OKT3 were used as negative and positive controls, respectively.

FIG. 8A depicts a chromium release assay (cytotoxicity assay). K562 parental line and K562/OKT3 were used as negative and positive controls, respectively. K562/EGFRvIII line is an engineered target line of 806CAR T cells expressing exogenous EGFRvIII. The effector to target cell ratio ranged from 30:1 to 1:1.

FIG. 8B depicts a chromium release assay (cytotoxicity assay) using different human donor cells as effector cells.

FIG. 8C depicts an incucyte analysis using human donor cells as effector cells against various target tumor cell lines.

FIG. 9 depicts a gene copy number analysis using droplet digital PCR. In this analyses, WPRE primers were used to target the lenti-viral backbone region integrated together with the gene of interest (806CAR-DHFRdm-EGFRt) in the genome.

FIG. 10A depicts an intracranial in vivo glioblastoma model.

FIG. 10B depicts the tumor bioluminescence signal quantification in mice. The codon optimized, short promoter, and long promoter sequences were transduced into T cells and studied in an intracranial NSG mouse model. T cells were used at a low dose (non-curative) to be able to detect the difference between the testing groups. Each testing group contained 5 mice. U87 glioma cells (806CAR target) expressing GFP:ffluc (GFP and firefly luciferase fusion protein) were injected intracranially (i.c.). A week later, T cells were i.c. injected. Bioluminescence images were taken at least once a week and the signal quantification is shown.

FIG. 10C depicts Kaplan Meier survival analysis in mice. The codon optimized, short promoter, and long promoter sequences were transduced into T cells and studied in an intracranial NSG mouse model. T cells were used at a low dose (non-curative) to be able to detect the difference between the testing groups. Each testing group contained 5 mice. The data disclosed herein depicts the percent of alive mice in each group per unit of time.

FIG. 10D depicts a tumor bioluminescence quantification.

FIG. 10E a depicts a Kaplan Meier survival analysis in mice. (*) mice were euthanized for tumor related symptoms including hunched posture, thinning of body weight, piloerection, lethargy, and labored breathing.

### DETAILED DESCRIPTION

Disclosed herein is a polynucleotide comprising a human codon-optimized sequence encoding a polypeptide an anti-EGFR chimeric antigen receptor (CAR). The codon-optimized sequence may be incorporated into a construct comprising an operably linked promoter, preferably an optimized promoter, spacer, intracellular signaling domain, transmembrane domain, selection marker, at least one self-cleaving peptides, and EFGRt, in order to optimize expression. This sequence may then be expressed in cells, such as T cells, for the treatment or inhibition of a cancer, such as glioblastoma, liquid tumors, or solid tumors.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, “a” or “an” may mean one or more than one.

As used herein, the term “about” has its usual meaning as understood by those skilled in the art and thus indicates that a value includes the inherent variation of error for the method being employed to determine a value, or the variation that exists among multiple determinations.

As used herein, the terms “modify” or “alter”, or any forms thereof, mean to modify, alter, replace, delete, substitute, remove, vary, or transform.

As used herein, the terms “function” and “functional” have their plain and ordinary meaning as understood in light of the specification, and refer to a biological, enzymatic, or therapeutic function.

As used herein, the terms “transduction” and “transfection” are used equivalently and the mean introducing a nucleic acid into a cell by an artificial method, including viral and non-viral methods.

As used herein, the term “isolated” has its plain and ordinary meaning as understood in light of the specification and refers to a substance and/or entity that has been (1) separated from at least some of the components with which it was associated when initially produced (whether in nature and/or in an experimental setting), and/or (2) produced, prepared, and/or manufactured by the hand of man. Nucleic acid molecules and proteins which have been “isolated” include nucleic acid molecules and proteins purified by standard purification methods. The term also embraces nucleic acid molecules and proteins prepared by recombinant expression in a host cell as well as chemically synthesized proteins and nucleic acids. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, or living cells which make up these living organisms, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The term “gene” as used herein have their plain and ordinary meaning as understood in light of the specification, and generally refers to a portion of a nucleic acid that encodes a protein or functional RNA; however, the term may optionally encompass regulatory sequences. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express or may be adapted to express using nucleic acid manipulation technology, proteins, polypeptides, domains, peptides, fusion proteins, mutants and/or such like.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g., plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 81, 82, 83, 84, 8580, 81, 82, 83, 84, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 81, 82, 83, 84, 85, 86, 87, 88, 89, 90, 95, 100, 150, 200, or 300 bases long, or any length in a range defined by any two of the aforementioned lengths, but generally not with a sequence in between that encodes for a functioning or catalytic polypeptide, protein, or protein domain.

As used herein, the term “codon” has its usual meaning as understood by those skilled in the art and refers to a sequence of three nucleotides, either RNA or DNA, that correspond to a particular amino acid or termination signal. Such codons can include, as non-limiting examples, the 61 natural occurring codons, 3 stop codons, start codon, and synthetic codons corresponding to a non-standard amino acid.

As used herein, the term “polynucleotide” has its usual meaning as understood by those skilled in the art and thus refers to a class of compounds that includes polydeoxynucleotides, polydeoxyribonucleotides, and polyribonucleotides. Thus, “polynucleotide” refers to an oligomer or polymer of ribonucleic acid (RNA) or deoxyribonucleic acid (DNA) or mimetics thereof, including reference to polynucleotides composed of naturally-occurring nucleobases, sugars and phosphodiester (PO) internucleoside (backbone) linkages as well as “modified” or substituted polynucleotides having non-naturally-occurring portions which function similarly.

The terms “peptide”, “polypeptide”, and “protein” used herein have their plain and ordinary meaning as understood in light of the specification and refer to macromolecules comprised of amino acids linked by peptide bonds. The term refers to both short chains (i.e. peptides, oligopeptides and oligomers) and to longer chains. The numerous functions of peptides, polypeptides, and proteins are known in the art, and include but are not limited to enzymes, structure, transport, defense, hormones, or signaling. Peptides resulting from translation of codons can include, as non-limiting examples, combinations of any of the 20 common amino acids, norleucine, ornithine, norvaline, homoserine, selenocysteine, pyrrolysine, non-coded amino acids, any of the over 140 amino acids found to occur in proteins, and synthetic amino acids constructed in a lab. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence. Proteins may contain amino acids other than the 20 gene encoded amino acids. Proteins include those modified by natural processes (e.g. processing and other post-translational modifications) and by chemical modification techniques. The same type of modification may be present in the same or varying degree at several sites in a given protein and a protein may contain many modifications. Modifications may occur in the peptide backbone, the amino acid side-chains, and the amino or carboxyl termini. Examples of modifications include acetylation; acylation; ADP-ribosylation; amidation; covalent attachment of flavin, a heme moiety, a nucleotide or nucleotide derivative, a lipid or lipid derivative, a carbohydrate, or phosphotidylinositol; cross-linking; cyclization; disulfide bond formation; demethylation, formation of covalent cross-links; glycosylation; hydroxylation; iodination; methylation; myristoylation; oxidation; proteoytic processing; phosphorylation; S-nitrosation; racemization; lipid attachment; sulfation, gamma-carboxylation of glutamic acid residues; or hydroxylation.

As used herein, the term “codon optimized” has its usual meaning as understood by those skilled in the art and refers to the optimization of a sequence for gene expression or protein production using molecular biology methods. For example, modifying a less-common codon with a more common codon may affect the half-life of the mRNA or alter its structure by introducing a secondary structure that interferes with translation of the message. In some embodiments, codon can be optimized using computer software and algorithms that predict the optimal sequences for expression efficiency. Nucleic acids can also be optimized for expression based on the cell type it will be incorporated in. Suitable host cells can include, as non-limiting examples, prokaryotic cells such as E coli. P. aeruginosa, B. subtilus, or V. natriegens, or eukaryotic cells such as S. cerevisiae, plant cells, insect cells, nematode cells, amphibian cells, fish cells, or mammalian cells, including human cells, such as but not limited to T cells. AU or a portion of a gene can be optimized. In some embodiments, the desired modulation of expression is achieved by optimizing essentially the entire gene. In other embodiments, the desired modulation will be achieved by optimizing part but not all of the gene.

As used herein, the term “promoter” has its usual meaning as understood by those skilled in the art and refers to a sequence of DNA to which proteins bind that regulates transcription of a polynucleotide. Examples of a promoter include, but are not limited to, EF1a, HTLV1, EF1a/HTLV (SEQ ID NO: 2), CMV, CAG, PGK, TRE, U6, UAS, T7, Sp6, lac, araBad, trp, or Ptac. Typically, a promoter is located in the 5′ region of a polynucleotide to be transcribed. More typically, promoters are defined as the region upstream of the first exon. A promoter can be any length. For example, short promoters, such as the EF1a core promoter, are between 100-300 base pairs in length, while long promoters, such as EF1a/HTLV, are between 400-1000 base pairs in length. In some embodiments, the promoter is naturally occurring. In other embodiments, the promoter is chemically synthesized according to techniques in common use. See, for example, Beaucage et al. (1981) Tet. Lett. 22:1859 and U.S. Pat. No. 4,668,777. A promoter can either regulate constitutive transcription, in which the gene product is continuously expressed, or inducible transcription, in which the expression of a gene product is influenced by certain conditions such as light, temperature, chemical concentration, protein concentration, conditions in an organism, cell, or organelle, etc. Promoters can be eukaryotic or prokaryotic, and modified to enhance the rate of expression or total copy number of its corresponding gene product. The promoter may also include at least one control element such as an upstream element. Such elements include UARs and optionally, other DNA sequences that affect transcription of a polynucleotide such as a synthetic upstream element. The term “promoter control element” as used herein describes elements that influence the activity of the promoter. Promoter control elements include transcriptional regulatory sequence determinants such as, but not limited to, enhancers, scaffold/matrix attachment regions, TATA boxes, transcription start locus control regions, UARs, URRs, other transcription factor binding sites and inverted repeats.

As used herein, the term “regulatory sequence” has its usual meaning as understood by those skilled in the art and refers to any nucleotide sequence that influences transcription or translation initiation and rate, or stability and/or mobility of a transcript or polypeptide product. Regulatory sequences include, but are not limited to, promoters, promoter control elements, protein binding sequences, 5′ and 3′ UTRs, transcriptional start sites, termination sequences, polyadenylation sequences, introns, certain sequences within amino acid coding sequences such as secretory signals, protease cleavage sites, etc.

As used herein, the term “operably linked” has its usual meaning as understood by those skilled in the art and denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous. In addition, in the context of a polypeptide, “operably linked” refers to a physical linkage (e.g., directly or indirectly linked) between amino acid sequences (e.g., different segments, regions, or domains) to provide for a described activity of the polypeptide. In the present disclosure, various segments, regions, or domains of the chimeric polypeptides of the disclosure may be operably linked to retain proper folding, processing, targeting, expression, binding, and other functional properties of the chimeric polypeptides in the cell. Unless stated otherwise, various regions, domains, and segments of the chimeric polypeptides of the disclosure are operably linked to each other. Operably linked regions, domains, and segments of the chimeric polypeptides of the disclosure may be contiguous or non-contiguous (e.g., linked to one another through a linker). DNA operably linked to a promoter is under transcriptional initiation regulation of the promoter or in functional combination therewith.

As used herein, the terms “self-cleavage peptide” or “self-cleaving peptide” have their usual meaning as understood by those skilled in the art and refer to a peptide sequence that undergoes cleavage of a peptide bond between two constituent amino acids, resulting in separation of the two proteins that flank the sequence. The cleavage is believed to be a result of a ribosomal “skipping” of the peptide bond formation between the C-terminal proline and glycine in the 2A peptide sequence. Polycistronic mRNA regulating elements were discovered with the investigation of viruses. The internal ribosome entry site (IRES) element was reported by two independent labs in 1988 studying poliovirus and encephalomyocarditis virus.

The term “internal ribosome entry site (IRES)” as used herein has its plain and ordinary meaning as understood in light of the specification and refers to the element that recruits the 40S subunit to promote translation of downstream mRNA. Similarly, the 2A family of self-cleaving peptides—P2A (SEQ ID NO: 3), E2A, F2A and T2A (SEQ ID NO: 4) were discovered in porcine teschovirus-1, equine rhinitis A, foot-and-mouth disease, and thosea asigna viruses, respectively. Both 2A and IRES elements allow multiple peptides to arise from a single strand of mRNA.

As used herein, the terms “selection marker” or “selectable marker” have their usual meaning as understood by those skilled in the art and refer to a gene which encodes a polypeptide that provides a phenotype to the cell containing the gene such that the phenotype allows either positive or negative, selection or screening of cells containing the selection marker gene. The selection marker gene may be used to distinguish between transformed and non-transformed cells or may be used to identify cells having undergone recombination or other kinds of genetic modifications. In some embodiments, the selection marker is co-expressed with the codon-optimized sequence corresponding to EGFR806CAR construct (SEQ ID NO: 1). Under this condition, the presence of a marker allows for the selection of cells containing the codon-optimized sequence. Non-limiting examples of selection markers include DHFRdm (SEQ ID NO: 5), DHFR, HER2T, MDR1, MRP1, O6-MGMT, cytidine deaminase, glutathione transferase Yc, or aldehyde dehydrogenase.

As used herein, the term “intracellular signaling domain” has its usual meaning as understood by those skilled in the art and refers to a portion of a protein facing the cellular interior which, when activated, positively or negatively regulates one or more signaling pathways in the host cell. Non-limiting examples of signaling pathways include proliferation, cytokine release, survival, cytotoxicity, phagocytosis, and metabolism. In some embodiments, the signaling domain includes a primary signaling domain. In some embodiments, the signaling domain includes a primary signaling domain and a secondary signaling domain. In some embodiments, the protein with said intracellular signaling domain is a transmembrane receptor, such as but not limited to EFGR, the EGFR variant EGFR806CAR construct, or the codon optimized EGFR variant EGFR806CAR construct. Non-limiting examples of an intracellular signaling domain include 41BB, CD3ξ OX40, CD27, or CD28. In some embodiments, the sequence contains two intracellular signaling domains, such as but not limited to 41BB and CD3ξ(SEQ ID NO: 7).

As used herein, the term “transmembrane domain” has its usual meaning as understood by those skilled in the art and refers to a portion of a protein that, when included, gets incorporated into the membrane region of a cell or organelle. In some embodiments, the membrane is the plasma membrane of a cell. Thus, a “transmembrane” protein is a protein containing the transmembrane domain. Most transmembrane domains form alpha helices. The transmembrane domain of a protein will span the entirety of the membrane. Proteins may contain multiple transmembrane domains, each of which will independently span the membrane. Other domains of a protein are referred to as either “extracellular” or “intracellular”, depending on their location relative to the cytoplasm and cell exterior. Non-limiting examples of transmembrane domains include CD28tm (SEQ ID NO. 8), CD8αtm, CD4tm, CD3ξtm, or the T cell receptor (TCR)-associated ξ chain.

As used herein, the terms “hinge” or “spacer” are used equivalently and have their usual meaning as understood by those skilled in the art and refer to a domain whose length, position and structure provide flexibility to the protein. In certain cases, the spacing of the antigen-recognition domain can be modified to reduce activation-induced cell death. The hinge region is found in IgG, IgA, and IgD immunoglobulin classes, such as but not limited to IgG4 (SEQ ID NO: 9).

### Cells, T Cells, and CAR-T Cells

The terms “individual”, “subject”, or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea or pigs.

The term “cell” as used herein has its plain and ordinary meaning as understood in light of the specification and can refer to any cell type. In some embodiments, said cells are mammalian cells. In some embodiments, said cells are human cells.

The term “immune cell” as used herein has its plain and ordinary meaning as understood in light of the specification and refers to any cell that participates in either the adaptive or innate immune response. Immune cells develop from stem cells in the bone marrow and become different types of white blood cells. Non-limiting examples of immune cells include memory cells, precursor T cells, precursor B cells, hematopoietic stem cells, myeloid progenitors, lymphoid progenitors, T cells, T helper cells, thymocytes, regulatory T cells, effector T cells, cytotoxic T lymphocytes, gamma/delta T cells, B cells, plasma cell, natural killer cells, antigen presenting cells, dendritic cells, macrophages, histiocytes, astrocytes, myeloid cells, monocytes, and granulocytes such as basophils, eosinophils, neutrophils, or mast cells.

The term “T cell” is used in its usual biological sense. Thus, a T cell is lymphocyte that participates in the adaptive immune system and contains a T-cell receptor on the cell surface. Non-limiting examples of a T cell include a CD4+ T cell, a CD8+ T cell, a CD8+ cytotoxic T cell, a naïve CD8+ T cell, a CD8+ memory T cell, a central memory CD8+ T cell, a regulatory CD8+ T cell, an IPS derived CD8+ T cell, an effector memory CD8+ T cell, a bulk CD8+ T cell, a CD4+T helper cell, a naïve CD4+ T cell, a CD4+ memory T cell, a central memory CD4+ T cell, a regulatory CD4+ T cell, an IPS derived CD4+ T cell, an effector memory CD4+ T cell, or a bulk CD4+ T cell.

The term “B cell” is used in its usual biological sense. Thus, a B cell is a lymphocyte that participates in the adaptive immune system and secretes antibodies. B cells can produce many types of antibodies and surface markers, such as but not limited to CD19, CD20, CD1d, CD5, CD19, CD20, CD21, CD22, CD23/Fc epsilon RII, CD24, CD25/IL-2 R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, Clq R1/CD93, CD84/SLAMF5, BAFF R TNFRSF13C, B220/CD45R, B7-1/CD80, B7-2/CD86, TNFSF7, TNFRSF5, ENPP-1, HVEM/TNFRSF14, BLIMP1/PRDM1, CXCR4, DEP-1/CD148, or EMMPRIN/CD147.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, humanized monoclonal antibodies, intracellular antibodies (“intrabodies”), Fv, Fab and F(ab)2, as well as single chain antibodies (scFv), camelid antibodies or humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen” as used herein has its plain and ordinary meaning as understood in light of the specification and refers to a compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes. Non-limiting examples of antigens include EGFR, EGFRvIII, HER2, MSLN, PSMA, CEA, GD2, IL13Ra2, GPC3, CAIX, L1-CAM, CA125, CD133, FAP, CTAG1B, MUC1, or FR-a.

The term “T-cell receptor” (or “TCR”) as used herein has its plain and ordinary meaning as understood in light of the specification and refers to a transmembrane protein found on the surface of T cells that is capable of recognizing an antigen. TCRs are described using the International Immunogenetics (1MGT) TCR nomenclature, and links to the IMGT public database of TCR sequences. Native T-cell receptors consist of two polypeptide chains, most commonly either alpha and beta chains, or gamma and delta chains. Broadly, each chain comprises variable, joining and constant regions. Each variable region comprises three CDRs (Complementarity Determining Regions) embedded in a framework sequence, one being the hypervariable region named CDR3. The joining regions of the TCR are similarly defined by the unique IMGT TRAJ and TRBJ nomenclature, and the constant regions by the IMGT TRAC and TRBC nomenclature. The unique sequences defined by the 1MGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

The terms “chimeric antigen receptors” (or “CAR”) T cells have their plain and ordinary meaning as understood in light of the specification and refer to T cells that have been genetically engineered to produce an artificial T-cell receptor for use in immunotherapy. Non-limiting examples of an artificial T-cell receptor include EGFR806CAR construct, or codon-optimized EGFR806CAR construct. A chimeric antigen receptor recognizes cell-surface tumor-associated antigen independent of human leukocyte antigen and employs one or more signaling molecules to activate genetically modified T cells for killing, proliferation, and cytokine production (Jena et al., 2010). The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. Adoptive transfer of T cells expressing CAR has shown promise in multiple clinical trials. It is now possible to use a modular approach to manufacture clinical grade genetically modified T cells. In specific embodiments, CARs direct specificity of the cell to a tumor associated antigen, for example. Non-limiting examples of tumor associated antigens include EGFR, EGFRvIII, HER2, MSLN, PSMA, CEA, GD2, IL13Ra2, GPC3, CAIX, L1-CAM, CA125, CD133, FAP, CTAGIB, MUC1, or FR-a. In some embodiments, CARs comprise an intracellular activation domain, a transmembrane domain, and an extracellular domain comprising a tumor associated antigen binding region. In certain cases, CARs comprise domains for additional co-stimulatory signaling, such as CD3-zeta, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

CAR T cells can also express a truncated version of the epidermal growth factor receptor (EGFRt) on the T cell surface (SEQ ID NO: 6). It has been shown that targeting EGFRt with the IgG1 monoclonal antibody cetuximab eliminates CD19 CAR T cells both early and late after adoptive transfer in mice, resulting in complete and permanent recovery of normal functional B cells, without tumor relapse. EGFRt can be incorporated into many clinical applications to regulate the survival of gene-engineered cells. See, for example, Paszkiewicz et al. (2016) J Clin Invest 126(11): 4262-4272.

### Cancer and Consequent Targeting by CAR-T Therapy

Further disclosed herein is a method of administering the novel polynucleotides and/or cells expressing the polynucleotides as a therapy against cancer. The term “cancer” is used in its usual biological sense. Thus, it can include the cancer of any cell type, such as but not limited to glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine sarcoma, vaginal cancer, vulvar cancer, Waldenstrom macroglobulinemia, Wilms tumor, solid tumor, or liquid tumor. The term “solid tumor” as used herein has its plain and ordinary meaning as understood in light of the specification and refers to an abnormal mass of tissue that does not contain liquid areas or cysts. Non-limiting examples of solid tumors include sarcomas, carcinomas, or lymphomas. Many cancer tissues can form solid tumors, such as but not limited to breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer.

The term “anti-cancer agent” as used herein has its plain and ordinary meaning as understood in light of the specification and refers to a small molecule, compound, protein, or other medicant that is used to treat, inhibit, or prevent cancer. Non-limiting examples of common classes of anti-cancer agents usable with any one or more of the alternatives described herein include alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators: immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-Jun-N-terminal kinase (JNK) inhibitors, mitogen-activated protein kinase (MAPK) inhibitors, MDM2 inhibitors, MEK inhibitors, ERK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinon, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin.

The terms “administration” or “administering” as used herein have their plain and ordinary meaning as understood in light of the specification and mean to provide or give a subject an agent, such as the composition disclosed herein, by any effective route. Exemplary routes of administration include, but are not limited to, oral, injection (such as intracranial, subcutaneous, intramuscular, intradermal, intraperitoneal, and intravenous), sublingual, rectal, transdermal, intranasal, vaginal, intraocular, or inhalation routes.

The terms “effective amount” or “effective dose” as used herein have their usual meaning as understood by those skilled in the art and refer to that amount of a recited composition or compound that results in an observable biological effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

As used herein, the term “pharmaceutically acceptable” has its usual meaning as understood by those skilled in the art and refers to carriers, diluents, excipients, and/or stabilizers that are nontoxic to the cell or mammal being exposed thereto at the dosages and concentrations employed or that have an acceptable level of toxicity. The terms “diluent,” “excipient,” and/or “carrier” as used herein have their usual meaning as understood by those skilled in the art and are include any and all solvents, dispersion media, coatings, antibacterial and antifungal agents, isotonic or absorption delaying agents, and the like, compatible with administration to humans, mice, rats, cats, dogs, or other vertebrate hosts.

Various pharmaceutically acceptable carriers, diluents, excipients, or combinations thereof can be incorporated into a pharmaceutical composition. In an embodiment, the pharmaceutically acceptable diluent, excipient, and/or carrier is one that is approved by a government regulatory agency or listed in the U.S. Pharmacopeia or other generally recognized pharmacopeia for use in animals, including humans as well as non-human mammals, such as cats, dogs, non-human primates, or mice. The pharmaceutically acceptable diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, or ethanol. A non-limiting example of a pharmaceutically acceptable carrier is an aqueous pH buffered solution. The pharmaceutically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, nonionic surfactants such as TWEEN®, polyethylene glycol (PEG), HEG, PLURONICS®, nanoparticles, nanosomes, micelles, lipid nanoparticles, or liposomes. Suitable pharmaceutical carriers also include any molecule or solvent that impacts the delivery, uptake, and metabolism of a drug, protein, or molecule. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

### Certain Sequences

Certain sequences useful with certain embodiments provided herein are listed in the following TABLE 1.

TABLE 1

| SEQ ID NO. | |
|---|---|
| Features | Sequence |
| SEQ ID NO: 01 | GACGTCCAGCTGCAAGAGTCTGGCCCTAGCCTGGTCAAGCC |
| Human codon- | TAGCCAGAGCCTGAGCCTGACATGTACCGTGACCGGCTACA |
| optimized | GCATCACCAGCGACTTCGCCTGGAACTGGATCAGACAGTTC |
| sequence | CCCGGCAACAAGCTGGAATGGATGGGCTACATCAGCTACA |
| encoding an scFv | GCGGCAACACCCGGTACAACCCCAGCCTGAAGTCCCGGAT |
| polypeptide | CTCCATCACCAGAGACACCAGCAAGAACCAGTTCTTCCTGC |
| capable of | AGCTGAACAGCGTGACCATCGAGGACACCGCCACCTACTA |
| specifically | CTGTGTGACAGCCGGCAGAGGCTTCCCTTATTGGGGACAGG |
| binding an EGFR | GAACCCTGGTCACAGTGTCTGCCGGAAGCACATCTGGCTCT |
| 806 epitope | GGCAAACCTGGATCTGGCGAGGGCTCTACCAAGGGCGACA |
| | TCCTGATGACACAGAGCCCCAGCAGCATGTCTGTGTCCCTG |
| | GGCGATACCGTGTCCATCACCTGTCACAGCAGCCAGGACAT |
| | CAACAGCAACATCGGCTGGCTGCAGCAGAGGCCTGGCAAG |
| | TCTTTTAAGGGCCTGATCTACCACGGCACCAACCTGGATGA |
| | TGAGGTGCCCAGCAGATTTTCCGGCTCTGGAAGCGGAGCCG |
| | ACTACTCCCTGACAATCAGCAGCCTGGAAAGCGAGGACTTC |
| | GCCGATTACTACTGCGTGCAGTACGCCCAGTTTCCTTGGAC |
| | CTTTGGCGGAGGCACAAAGCTGGAAATCAAGCGC |
| SEQ ID NO: 02 | GGATCTGCGATCGCTCCGGTGCCCGTCAGTGGGCAGAGCGC |
| EF1a/HTLV | ACATCGCCCACAGTCCCCGAGAAGTTGGGGGGAGGGGTCG |
| promoter | GCAATTGAACCGGTGCCTAGAGAAGGTGGCGCGGGGTAAA |
| | CTGGGAAAGTGATGTCGTGTACTGGCTCCGCCTTTTTCCCG |
| | AGGGTGGGGGAGAACCGTATATAAGTGCAGTAGTCGCCGT |
| | GAACGTTCTTTTTCGCAACGGGTTTGCCGCCAGAACACAGC |
| | TGAAGCTTCGAGGGGCTCGCATCTCTCCTTCACGCGCCCGC |
| | CGCCCTACCTGAGGCCGCCATCCACGCCGGTTGAGTCGCGT |
| | TCTGCCGCCTCCCGCCTGTGGTGCCTCCTGAACTGCGTCCG |
| | CCGTCTAGGTAAGTTTAAAGCTCAGGTCGAGACCGGGCCTT |
| | TGTCCGGCGCTCCCTTGGAGCCTACCTAGACTCAGCCGGCT |
| | CTCCACGCTTTGCCTGACCCTGCTTGCTCAACTCTACGTCTT |
| | TGTTTCGTTTTCTGTTCTGCGCCGTTACAGATCCAAGCTGTG |
| | ACCGGCGCCTAC |
| SEQ ID NO: 03 | GGAAGCGGCGCCACAAATTTCAGCCTGCTGAAACAGGCCG |
| Encodes a | GCGACGTGGAAGAGAACCCTGGACCT |
| polypeptide | |
| comprising self- | |
| cleavage peptide | |
| P2A | |
| SEQ ID NO: 04 | GGCGGAGGCGAAGGCAGAGGTTCTCTGCTTACATGCGGAG |
| Encodes a | ATGTGGAAGAAAATCCCGGGCCT |
| polypeptide | |
| comprising self- | |
| cleavage peptide | |
| T2A. | |
| SEQ ID NO: 05 | ATGGTCGGAAGCCTGAACTGCATCGTGGCCGTGTCTCAGAA |
| Encodes a | CATGGGCATCGGCAAGAACGGCGACTTCCCTTGGCCTCCTC |
| polypeptide | TGAGAAACGAGAGCCGGTACTTCCAGCGGATGACCACCAC |
| comprising | AAGCAGCGTGGAAGGCAAGCAGAACCTGGTCATCATGGGC |
| selection marker | AAGAAAACCTGGTTCAGCATCCCTGAGAAGAACAGACCCC |
| DHFRdm | TGAAGGGCAGAATCAACCTGGTGCTGAGCAGAGAGCTGAA |
| | AGAGCCTCCTCAGGGCGCCCACTTTCTGAGCAGATCTCTGG |
| | ACGATGCCCTGAAGCTGACCGAGCAACCTGAGCTGGCCAA |
| | CAAGGTGGACATGGTCTGGATCGTTGGCGGCAGCAGCGTG |
| | TACAAAGAAGCCATGAATCACCCCGGCCACCTGAAACTGTT |
| | CGTGACCAGAATCATGCAGGACTTCGAGAGCGACACATTCT |
| | TCCCAGAGATCGACCTGGAAAAGTACAAACTGCTGCCTGA |
| | GTACCCCGGCGTGCTGAGCGACGTGCAAGAAGAGAAAGGC |
| | ATCAAGTACAAGTTCGAGGTGTACGAGAAGAACGAC |
| SEQ ID NO: 06 | CGGAAAGTGTGCAACGGCATCGGAATCGGCGAGTTCAAGG |
| Codon-optimized | ACAGCCTGAGCATCAACGCCACCAACATCAAGCACTTCAA |
| sequence | GAACTGCACCAGCATCAGCGGCGACCTGCACATTCTGCCTG |
| encoding a | TGGCCTTTAGAGGCGACAGCTTCACCCACACACCTCCACTG |
| polypeptide | GATCCCCAAGAGCTGGATATCCTGAAAACCGTGAAAGAGA |
| comprising a | TCACCGGATTTCTGTTGATCCAGGCTTGGCCCGAGAACCGG |
| truncated EGFR | ACAGATCTGCACGCCTTCGAGAACCTCGAGATCATCAGAG |
| (EGFRt). | GCCGGACCAAGCAGCACGGCCAGTTTTCTCTGGCCGTGGTG |
| | TCCCTGAATATCACCTCTCTGGGCCTGCGCAGCCTGAAAGA |
| | AATCTCCGATGGCGACGTGATCATCAGCGGAAACAAGAAC |
| | CTGTGCTACGCCAACACCATCAACTGGAAGAAGCTGTTCGG |
| | CACCTCCGGCCAGAAAACAAAGATCATCTCCAACCGGGGC |
| | GAGAACTCCTGCAAGGCTACAGGCCAAGTGTGCCACGCTCT |
| | GTGTAGCCCTGAAGGCTGTTGGGGACCCGAGCCTAGAGATT |
| | GCGTGTCCTGCAGAAACGTGTCCCGGGGCAGAGAATGCGT |
| | GGACAAGTGCAATCTGCTCGAGGGCGAGCCACGCGAGTTC |
| | GTGGAAAACAGCGAGTGCATCCAGTGTCACCCCGAGTGCC |
| | TGCCTCAGGCCATGAACATCACATGCACCGGAAGAGGCCC |
| | CGACAACTGTATCCAGTGCGCCCACTATATCGACGGCCCTC |
| | ACTGCGTGAAAACCTGTCCTGCTGGCGTGATGGGAGAGAA |
| | CAACACCCTCGTGTGGAAGTACGCCGATGCCGGACATGTGT |
| | GCCACCTGTGTCACCCTAATTGCACCTACGGCTGTACAGGC |
| | CCAGGACTGGAAGGCTGCCCTACAAACGGACCTAAGATCC |
| | CCAGCATTGCCACCGGCATGGTTGGAGCCCTGCTGCTTCTG |
| | CTGGTGGTGGCCCTTGGAATCGGACTGTTTATG |
| SEQ ID NO: 07 | AAGCGGGGCAGAAAGAAGCTGCTGTACATCTTCAAGCAGC |
| Encodes a | CCTTCATGCGGCCCGTGCAGACCACACAAGAGGAAGATGG |
| polypeptide | CTGCTCCTGCAGATTCCCCGAGGAAGAAGAAGGCGGCTGC |
| comprising the | GAGCTGAGAGTGAAGTTCAGCAGATCCGCCGACGCTCCTG |
| intracellular | CCTATCAGCAGGGACAGAACCAGCTGTACAACGAGCTGAA |
| signaling domains | CCTGGGGAGAAGAGAAGAGTACGACGTGCTGGACAAGCGG |
| 41BB and CD34. | AGAGGCAGAGATCCTGAGATGGGCGGCAAGCCCAGACGGA |
| | AGAATCCTCAAGAGGGCCTGTATAATGAGCTGCAGAAAGA |
| | CAAGATGGCCGAGGCCTACAGCGAGATCGGAATGAAGGGC |
| | GAGCGCAGAAGAGGCAAGGGACACGATGGACTGTACCAGG |
| | GACTGAGCACCGCCACAAAGGACACCTATGACGCCCTGCA |
| | CATGCAGGCCCTTCCACCTAGA |
| SEQ ID NO: 08 | ATGTTCTGGGTGCTCGTGGTTGTTGGCGGAGTGCTGGCCTG |
| Encodes a | TTATAGCCTGCTTGTGACCGTGGCCTTCATCATCTTTTGGGT |
| polypeptide | C |
| comprising the | |
| transmembrane | |
| domain CD28tm | |
| SEQ ID NO: 09 | GAGTCTAAGTACGGCCCTCCTTGTCCTCCATGTCCT |
| Encodes a | |
| polypeptide | |
| comprising the | |
| hinge region of | |
| IgG4. | |
| SEQ ID NO: 10 | GGATCTGCGATCGCTCCGGTGCCCGTCAGTGGGCAGAGCGC |
| EF1a/HTLV | ACATCGCCCACAGTCCCCGAGAAGTTGGGGGGAGGGGTCG |
| promoter, and | GCAATTGAACCGGTGCCTAGAGAAGGTGGCGCGGGGTAAA |
| sequence | CTGGGAAAGTGATGTCGTGTACTGGCTCCGCCTTTTTCCCG |
| encoding a | AGGGTGGGGGAGAACCGTATATAAGTGCAGTAGTCGCCGT |
| polypeptide | GAACGTTCTTTTTCGCAACGGGTTTGCCGCCAGAACACAGC |
| comprising | TGAAGCTTCGAGGGGCTCGCATCTCTCCTTCACGCGCCCGC |
| codon-optimized | CGCCCTACCTGAGGCCGCCATCCACGCCGGTTGAGTCGCGT |
| human | TCTGCCGCCTCCCGCCTGTGGTGCCTCCTGAACTGCGTCCG |
| EGFR806CAR | CCGTCTAGGTAAGTTTAAAGCTCAGGTCGAGACCGGGCCTT |
| scFv, IgG4, | TGTCCGGCGCTCCCTTGGAGCCTACCTAGACTCAGCCGGCT |
| CD28tm, 41BB, | CTCCACGCTTTGCCTGACCCTGCTTGCTCAACTCTACGTCTT |
| CD3ζ, P2A, | TGTTTCGTTTTCTGTTCTGCGCCGTTACAGATCCAAGCTGTG |
| DHFRdm, T2A, | ACCGGCGCCTACGGCTAGCGCCGCCACCATGTTGCTGCTGG |
| and EGFRt | TTACATCTCTGCTGCTGTGCGAGCTGCCCCATCCTGCCTTTC |
| | TGCTGATCCCTGACGTCCAGCTGCAAGAGTCTGGCCCTAGC |
| | CTGGTCAAGCCTAGCCAGAGCCTGAGCCTGACATGTACCGT |
| | GACCGGCTACAGCATCACCAGCGACTTCGCCTGGAACTGG |
| | ATCAGACAGTTCCCCGGCAACAAGCTGGAATGGATGGGCT |
| | ACATCAGCTACAGCGGCAACACCCGGTACAACCCCAGCCT |
| | GAAGTCCCGGATCTCCATCACCAGAGACACCAGCAAGAAC |
| | CAGTTCTTCCTGCAGCTGAACAGCGTGACCATCGAGGACAC |
| | CGCCACCTACTACTGTGTGACAGCCGGCAGAGGCTTCCCTT |
| | ATTGGGGACAGGGAACCCTGGTCACAGTGTCTGCCGGAAG |
| | CACATCTGGCTCTGGCAAACCTGGATCTGGCGAGGGCTCTA |
| | CCAAGGGCGACATCCTGATGACACAGAGCCCCAGCAGCAT |
| | GTCTGTGTCCCTGGGCGATACCGTGTCCATCACCTGTCACA |
| | GCAGCCAGGACATCAACAGCAACATCGGCTGGCTGCAGCA |
| | GAGGCCTGGCAAGTCTTTTAAGGGCCTGATCTACCACGGCA |
| | CCAACCTGGATGATGAGGTGCCCAGCAGATTTTCCGGCTCT |
| | GGAAGCGGAGCCGACTACTCCCTGACAATCAGCAGCCTGG |
| | AAAGCGAGGACTTCGCCGATTACTACTGCGTGCAGTACGCC |
| | CAGTTTCCTTGGACCTTTGGCGGAGGCACAAAGCTGGAAAT |
| | CAAGCGCGAGTCTAAGTACGGCCCTCCTTGTCCTCCATGTC |
| | CTATGTTCTGGGTGCTCGTGGTTGTTGGCGGAGTGCTGGCC |
| | TGTTATAGCCTGCTTGTGACCGTGGCCTTCATCATCTTTTGG |
| | GTCAAGCGGGGCAGAAAGAAGCTGCTGTACATCTTCAAGC |
| | AGCCCTTCATGCGGCCCGTGCAGACCACACAAGAGGAAGA |
| | TGGCTGCTCCTGCAGATTCCCCGAGGAAGAAGAAGGCGGC |
| | TGCGAGCTGAGAGTGAAGTTCAGCAGATCCGCCGACGCTC |
| | CTGCCTATCAGCAGGGACAGAACCAGCTGTACAACGAGCT |
| | GAACCTGGGGAGAAGAGAAGAGTACGACGTGCTGGACAAG |
| | CGGAGAGGCAGAGATCCTGAGATGGGCGGCAAGCCCAGAC |
| | GGAAGAATCCTCAAGAGGGCCTGTATAATGAGCTGCAGAA |
| | AGACAAGATGGCCGAGGCCTACAGCGAGATCGGAATGAAG |
| | GGCGAGCGCAGAAGAGGCAAGGGACACGATGGACTGTACC |
| | AGGGACTGAGCACCGCCACAAAGGACACCTATGACGCCCT |
| | GCACATGCAGGCCCTTCCACCTAGAGGAAGCGGCGCCACA |
| | AATTTCAGCCTGCTGAAACAGGCCGGCGACGTGGAAGAGA |
| | ACCCTGGACCTATGGTCGGAAGCCTGAACTGCATCGTGGCC |
| | GTGTCTCAGAACATGGGCATCGGCAAGAACGGCGACTTCC |
| | CTTGGCCTCCTCTGAGAAACGAGAGCCGGTACTTCCAGCGG |
| | ATGACCACCACAAGCAGCGTGGAAGGCAAGCAGAACCTGG |
| | TCATCATGGGCAAGAAAACCTGGTTCAGCATCCCTGAGAA |
| | GAACAGACCCCTGAAGGGCAGAATCAACCTGGTGCTGAGC |
| | AGAGAGCTGAAAGAGCCTCCTCAGGGCGCCCACTTTCTGA |
| | GCAGATCTCTGGACGATGCCCTGAAGCTGACCGAGCAACCT |
| | GAGCTGGCCAACAAGGTGGACATGGTCTGGATCGTTGGCG |
| | GCAGCAGCGTGTACAAAGAAGCCATGAATCACCCCGGCCA |
| | CCTGAAACTGTTCGTGACCAGAATCATGCAGGACTTCGAGA |
| | GCGACACATTCTTCCCAGAGATCGACCTGGAAAAGTACAA |
| | ACTGCTGCCTGAGTACCCCGGCGTGCTGAGCGACGTGCAAG |
| | AAGAGAAAGGCATCAAGTACAAGTTCGAGGTGTACGAGAA |
| | GAACGACGGCGGAGGCGAAGGCAGAGGTTCTCTGCTTACA |
| | TGCGGAGATGTGGAAGAAAATCCCGGGCCTATGCTGCTGCT |
| | CGTGACAAGCCTGCTCCTGTGTGAACTCCCTCATCCAGCTT |
| | TTCTGCTCATTCCCCGGAAAGTGTGCAACGGCATCGGAATC |
| | GGCGAGTTCAAGGACAGCCTGAGCATCAACGCCACCAACA |
| | TCAAGCACTTCAAGAACTGCACCAGCATCAGCGGCGACCT |
| | GCACATTCTGCCTGTGGCCTTTAGAGGCGACAGCTTCACCC |
| | ACACACCTCCACTGGATCCCCAAGAGCTGGATATCCTGAAA |
| | ACCGTGAAAGAGATCACCGGATTTCTGTTGATCCAGGCTTG |
| | GCCCGAGAACCGGACAGATCTGCACGCCTTCGAGAACCTC |
| | GAGATCATCAGAGGCCGGACCAAGCAGCACGGCCAGTTTT |
| | CTCTGGCCGTGGTGTCCCTGAATATCACCTCTCTGGGCCTG |
| | CGCAGCCTGAAAGAAATCTCCGATGGCGACGTGATCATCA |
| | GCGGAAACAAGAACCTGTGCTACGCCAACACCATCAACTG |
| | GAAGAAGCTGTTCGGCACCTCCGGCCAGAAAACAAAGATC |
| | ATCTCCAACCGGGGCGAGAACTCCTGCAAGGCTACAGGCC |
| | AAGTGTGCCACGCTCTGTGTAGCCCTGAAGGCTGTTGGGGA |
| | CCCGAGCCTAGAGATTGCGTGTCCTGCAGAAACGTGTCCCG |
| | GGGCAGAGAATGCGTGGACAAGTGCAATCTGCTCGAGGGC |
| | GAGCCACGCGAGTTCGTGGAAAACAGCGAGTGCATCCAGT |
| | GTCACCCCGAGTGCCTGCCTCAGGCCATGAACATCACATGC |
| | ACCGGAAGAGGCCCCGACAACTGTATCCAGTGCGCCCACT |
| | ATATCGACGGCCCTCACTGCGTGAAAACCTGTCCTGCTGGC |
| | GTGATGGGAGAGAACAACACCCTCGTGTGGAAGTACGCCG |
| | ATGCCGGACATGTGTGCCACCTGTGTCACCCTAATTGCACC |
| | TACGGCTGTACAGGCCCAGGACTGGAAGGCTGCCCTACAA |
| | ACGGACCTAAGATCCCCAGCATTGCCACCGGCATGGTTGGA |
| | GCCCTGCTGCTTCTGCTGGTGGTGGCCCTTGGAATCGGACT |
| | GTTTATGTAG |

### Certain Polynucleotides

Some embodiments of the methods and compositions provided herein include polynucleotides comprising a sequence encoding a chimeric antigen receptor (CAR) capable of specifically binding EGFR. In some embodiments, the sequence encoding the CAR is codon-optimized for expression in a human cell. In some embodiments, the sequence encoding the CAR is codon-optimized for expression in a human cell is operably linked to a promoter comprising an Ela promoter, such as an Ela minimal promoter. The sequence encoding the CAR is codon-optimized for expression in a human cell is operably linked to a promoter comprising an Ela promoter, such as an Ela minimal promoter, and a HTLV element.

Some embodiments include a polynucleotide comprising a human codon-optimized sequence encoding a polypeptide comprising an EGFR806CAR scFv. In some embodiments, the human codon-optimized sequence comprises the sequence set forth in SEQ ID NO: 1. Some embodiments also include a promoter operably linked to the human codon-optimized sequence. In some embodiments, the promoter comprises an EF1a sequence, or an EF1a/HTLV sequence, preferably a human EF1a sequence, or a human EF1a/HTLV sequence. In some embodiments, the promoter comprises the sequence set forth in SEQ ID NO: 2.

Some embodiments also include at least one sequence encoding a self-cleavage peptide or an IRES, preferably wherein said sequence encoding said self-cleavage peptide or said IRES is codon-optimized for expression in humans. In some embodiments, the self-cleavage peptide is a 2A self-cleaving peptide, such as P2A or T2A or both. In some embodiments, the sequence encoding the self-cleavage peptide comprises the sequences set forth in SEQ ID NO: 3 or SEQ ID NO:4

Some embodiments also include a sequence encoding one or more selection markers, wherein said sequence encoding said one or more selection markers is preferably codon-optimized for expression in humans. In some embodiments, the one or more selection markers comprises DHFRdm. In some embodiments, the sequence encoding said one or more selection markers comprises the sequence set forth in SEQ ID NO: 5.

Some embodiments also include a sequence encoding a truncated EGFR polypeptide (EGFRt), preferably wherein said sequence encoding EGFRt is codon optimized for expression in humans. In some embodiments, the sequence encoding said EGFRt comprises the sequence set forth in SEQ ID NO: 6.

Some embodiments also include a sequence encoding one or more intracellular signaling domains, preferably wherein said sequence encoding said one or more intracellular signaling domains is codon-optimized for expression in humans. In some embodiments, the intracellular signaling domains comprise 41BB or CD3 or both. In some embodiments, the sequence encoding said one or more intracellular signaling domains comprises the sequence set forth in SEQ ID NO: 7.

Some embodiments also include a sequence encoding a transmembrane domain, preferably wherein said sequence encoding said transmembrane domain is codon-optimized for expression in humans. In some embodiments, the transmembrane domain comprises CD28tm. In some embodiments, the sequence encoding said transmembrane domain comprises the sequence set forth in SEQ ID NO: 8.

Some embodiments also include a sequence encoding a spacer, preferably wherein said sequence encoding said spacer is codon-optimized for expression in humans. In some embodiments, the spacer comprises a portion of IgG4, such as a hinge region of IgG4. In some embodiments, the sequence encoding a spacer is set forth in SEQ ID NO: 9.

In some embodiments, the sequence of the polynucleotide is set forth in SEQ ID NO: 10.

### Certain Cells

Some embodiments of the methods and compositions provided herein include cells comprising any one of the polynucleotides provided herein. In some embodiments, the cell is an immune cell. In some embodiments, the cell is a precursor T cell, or a hematopoietic stem cell. In some embodiments, the cell is a T cell, a B cell, a natural killer cell, an antigen presenting cell, a dendritic cell, a macrophage, or a granulocyte such as a basophil, an eosinophil, a neutrophil, or a mast cell. In some embodiments, the cell is a CD4+ T cell or a CD8+ T cell. In some embodiments, the cell is a CD8+ cytotoxic T cell selected from the group consisting of a naïve CD8+ T cell, a CD8+ memory T cell, a central memory CD8+ T cell, a regulatory CD8+ T cell, an IPS derived CD8+ T cell, an effector memory CD8+ T cell, and a bulk CD8+ T cell. In some embodiments, the cell is a CD4+T helper cell selected from the group consisting of a naïve CD4+ T cell, a CD4+ memory T cell, a central memory CD4+ T cell, a regulatory CD4+ T cell, an IPS derived CD4+ T cell, an effector memory CD4+ T cell, and a bulk CD4+ T cell. In some embodiments, the cell is allogenic to a subject, or is autologous to a subject. In some embodiments, the cell is ex vivo. In some embodiments, the cell is in vivo. In some embodiments, the cell is mammalian. In some embodiments, the cell is human.

In some embodiments, the cell also expresses an antibody or binding fragment thereof or scFv specific for a B cell specific cell surface molecule, such as CD19, CD20, CD1d, CD5, CD19, CD20, CD21, CD22, CD23/Fc epsilon RII, CD24, CD25/IL-2 R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, Clq R1/CD93, CD84/SLAMF5, BAFF R TNFRSF13C, B220/CD45R, B7-1/CD80, B7-2/CD86, TNFSF7, TNFRSF5, ENPP-1, HVEM/TNFRSF14, BLIMP1/PRDM1, CXCR4, DEP-1/CD148, or EMMPRIN/CD147.

### Certain Therapies

Some embodiments of the methods and compositions provided herein include therapies inhibiting, ameliorating, or treating a cancer in a subject in need thereof. Some embodiments include methods of inhibiting ameliorating, or treating a cancer in a subject in need thereof, preferably a human, comprising administering any one of the polynucleotides provided herein, or any one of the cells provided herein containing such polynucleotides to said subject.

Some embodiments include use of any one of the polynucleotides provided herein, or any one of the cells provided herein containing such polynucleotides as a medicament, or in the preparation of such a medicament.

In some embodiments, the cancer is a leukemia, a lymphoma, a hematological tumor, a liquid tumor, or a solid tumor. In some embodiments, the solid tumor is selected from the group consisting of a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas and ovarian cancer. In some embodiments, the cancer is glioblastoma. In some embodiments, the administration is conducted by intracranial injection.

As disclosed herein, the inventors discovered that integrating a human codon-optimized EGFR806CAR construct sequence into T cells dramatically enhanced expression and conferred a therapeutic benefit for cancer therapies. Specific examples of these findings are described in the examples below.

### EXAMPLES

### Example 1-Design of a Human Codon Optimized EGFR806CAR Construct and Insertion into T Cells

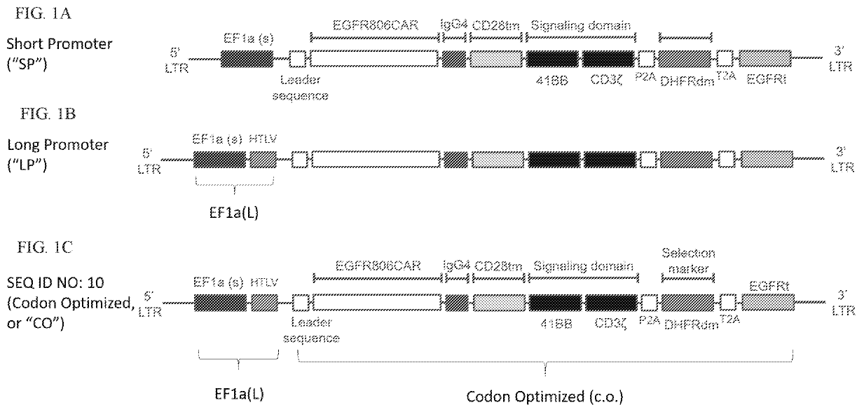

As disclosed herein, the inventors designed three novel sequences of EGFR806CAR construct to test for expression in T cells and effectiveness in cancer therapy. These constructs included the EGFR806CAR construct controlled by a short EF1a promoter (253 nucleotides) (FIG. 1A), also known herein as the “HIV7.2 construct”; as well as the EGFR806CAR construct controlled by a long EF1a and HTLV promoter (544 nucleotides) (FIG. 1B), also known herein as the “HIV7.3 construct”. The third construct was controlled by the long EF1a and HTLV promoter, and the entire open reading frame was codon optimized for expression in humans (FIG. 1C) (SEQ ID NO: 10), also known herein as the “coHIV7.2 construct”. Codon optimization was done using the GeneArt online Algorithm.

The open reading frames contained a leader sequenced, the EGFR806CAR construct, the hinge region of TgG4, the transmembrane domain CD28tm, the signaling domains 41BB and CD3ξ, the self-cleaving peptides P2A and T2A, the drug selection marker DHFRdm, and the surface marker EGFRt. This entire region was codon optimized for expression in humans in SEQ ID NO: 10.

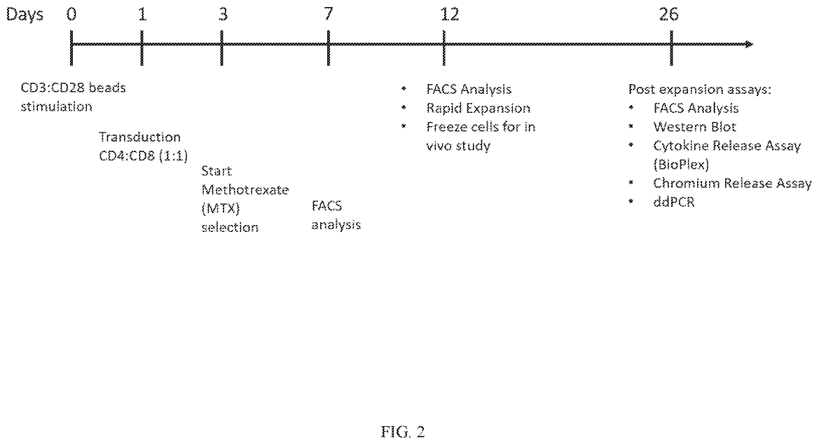

As disclosed herein, the inventors inserted the three constructs into human primary T cells (FIG. 2). The inventors stimulated polyclonal expansion of primary human T cells with CD3:CD28 beads. After 24 hours, lentiviral particles containing one of the three sequences were mixed with protamine sulfate and added to the primary T cells composed of CD4 and CD8 cells at 1:1 ratio. “Mock cells”, to which only protamine sulfate was added, is referenced hereafter as the negative control. At a total of three days after stimulation, the inventors began methotrexate (MTX) selection. At a total of seven days after stimulation, inventors began fluorescence-activated cell sorting (FACS) flow cytometry analysis. At a total of twelve days after stimulation, the inventors continued FACS analysis, then performed rapid expansion and froze cells for future in vivo studies. At a total of 26 days after stimulation, the inventors performed post-expansion assays, including FACS analysis, western blot analysis, cytokine release, chromium release, and ddPCR.

### Example 2-Human Codon Optimized EGFR806CAR Construct had Enhanced Expression in T Cells

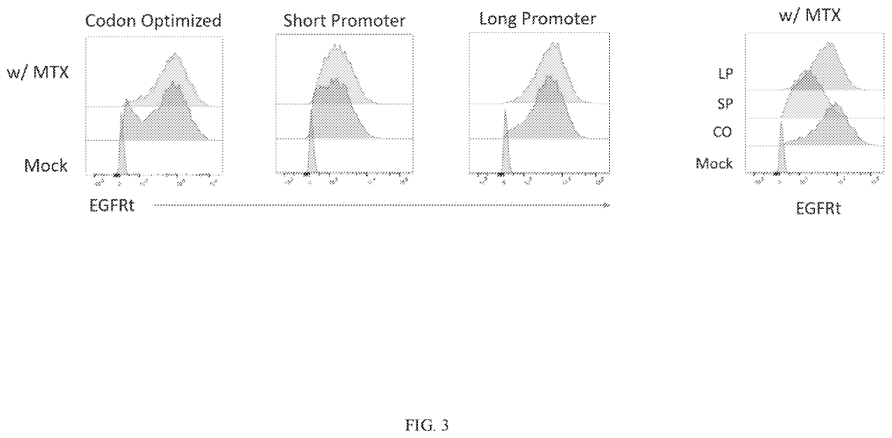

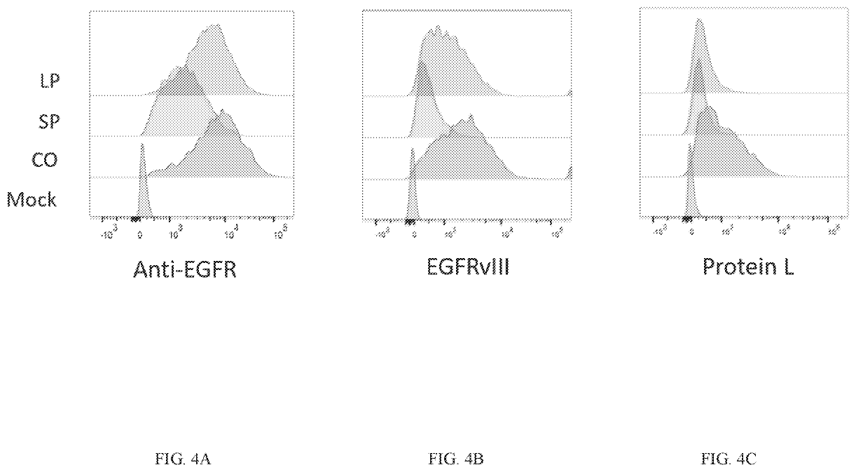

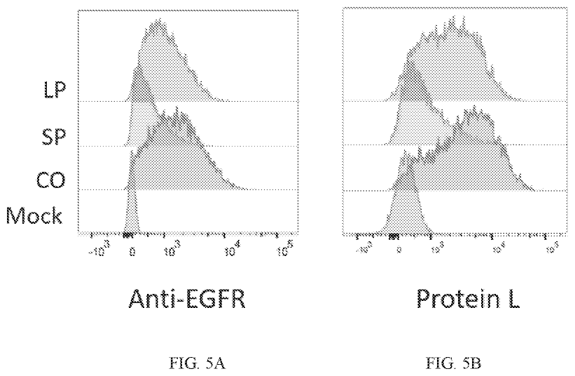

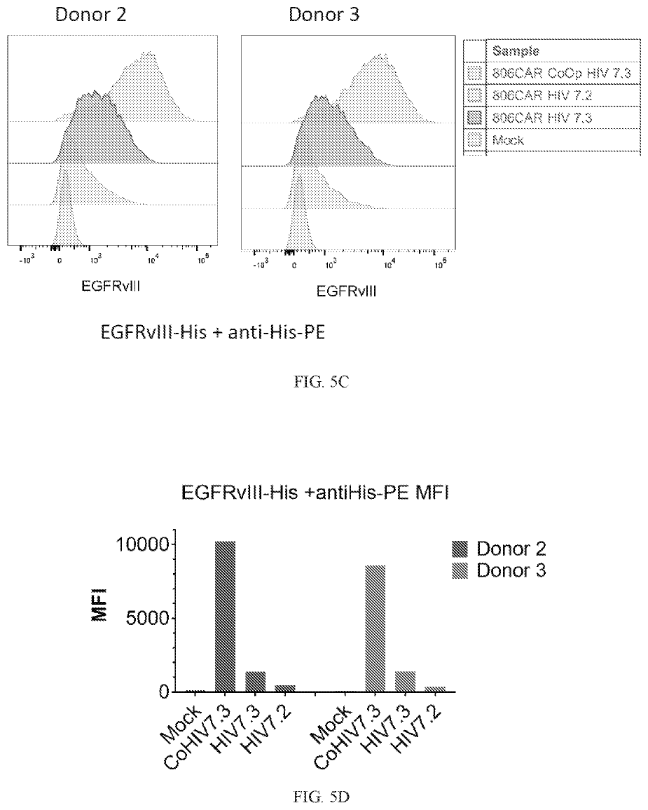

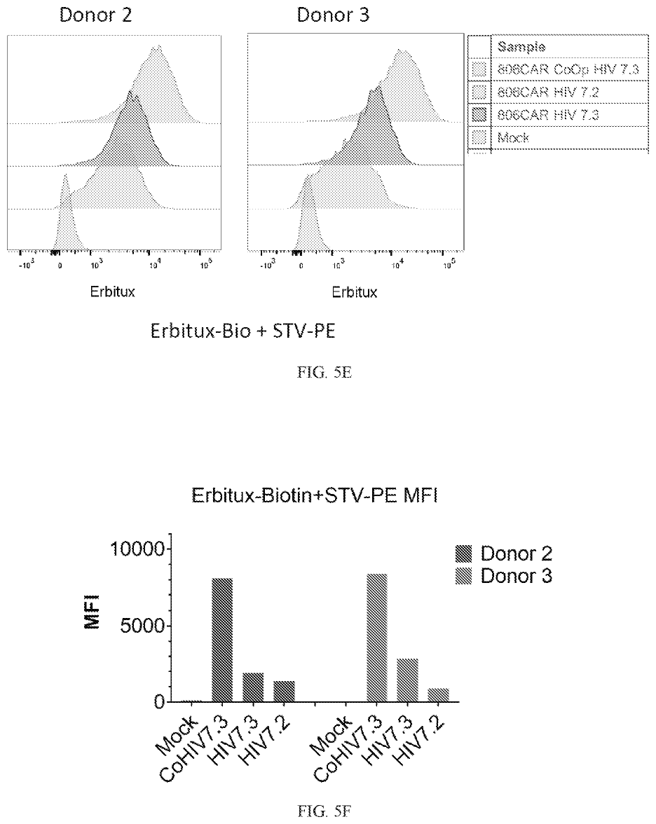

As disclosed herein, the inventors established the expression of EGFR806CAR construct from all three sequence variants and a mock control in human primary T cells. Six days post transduction and four days post methotrexate (MTX) selection, the primary human T cells were stained with anti-EGFR-Biotin and Streptavidin-APC (FIG. 3). Surprisingly, the inventors found that the long promoter enhanced expression, and that the human codon optimized variant further dramatically enhanced expression in T cells. Eleven days post transduction, this trend of significantly enhanced expression of the truncated EGFR (EGFRt) continued (FIG. 4A). Also tested was CAR expression through staining with either EGFRvIII-his and anti-his-APC (FIG. 4B), or Protein L-Biotin and Streptavidin-BV405 (FIG. 4C). Both EGFRvIII (CAR antigen) and Protein L bind to 806CAR Similar to EGFRt expression, CAR expression was shown to be significantly higher in T cells containing the human codon optimized sequence. Even 7 days post rapid expansion, the human codon optimized sequence showed significantly higher EGFRt and CAR expression (FIG. 5A, FIG. 5B).

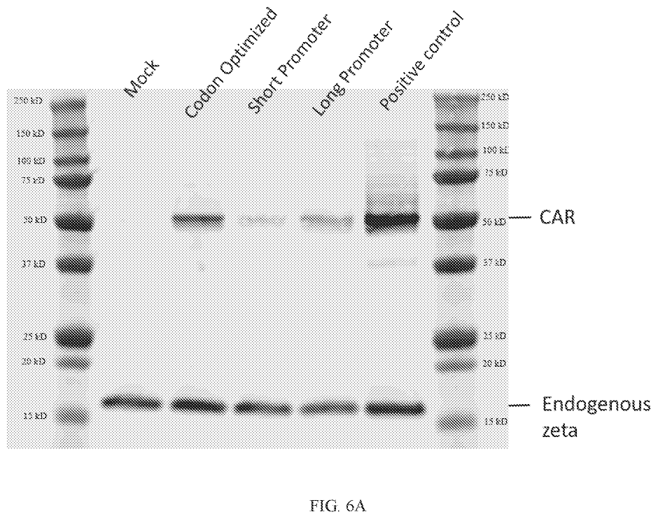

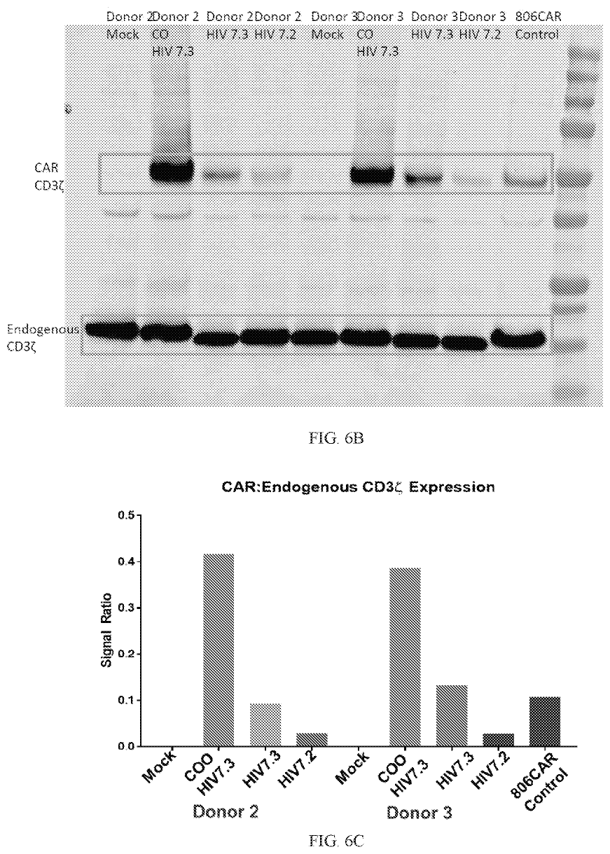

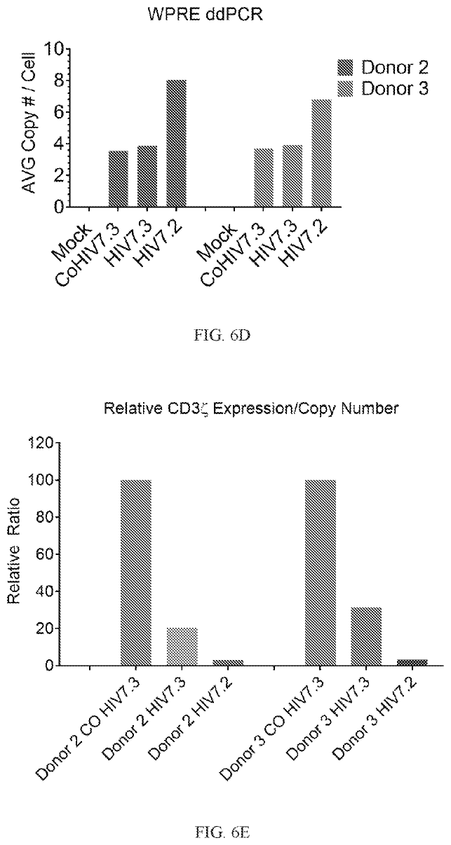

Also disclosed herein, the inventors tested expression and protein processing of the CARs in parallel to flow cytometry using western blot analysis (FIG. 6A). Protein lysate were collected, and the western blot ran using standard methods. The western blot was visualized using an anti-CD3 zeta antibody. The molecular weight of the CAR is about 50 kD and the endogenous zeta is about 15 kD. The positive control is H9 cells expressing 806CAR (>99% pure by flow). The results of the western blot were the same as those shown by flow cytometry, expression was enhanced slightly by introducing a long promoter, and significantly enhanced by introducing a long promoter and codon optimized open reading frame.

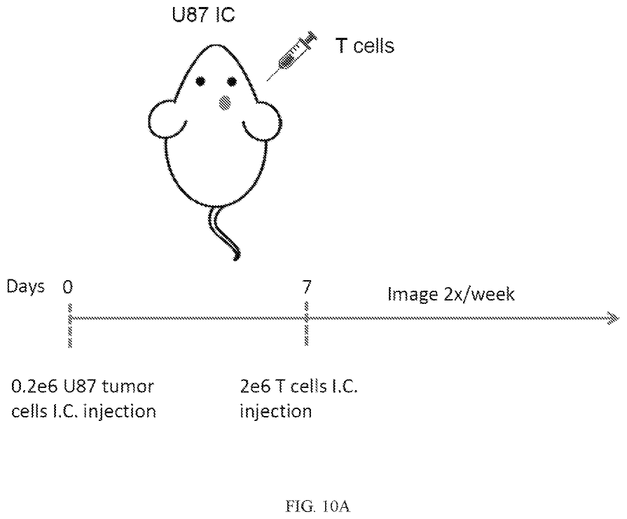

As a control, the inventors also assessed whether the high expression from the human codon optimized EGFR806CAR construct was caused by a high gene copy number in the cells. They tested this using droplet digital (dd) PCR under standard methods (FIG. 9). ddPCR was conducted using WPRE primers that target the lenti-viral backbone region integrated together with the gene of interest (806CAR-DHFRdm-EGFRt) in the genome. As shown in the drawing, the average copy number per cell was surprisingly lower in the human codon optimized EGFR806CAR construct compared with the long and short promoter sequences. This demonstrated that the effect shown is from high gene expression, rather than the genetic copy number.

In additional experiments, human T cells from donors were transduced with constructs depicted in FIG. 1A, FIG. 1B, and FIG. 1C, and selected with methotrexate. At day 14, the cells were stained with an 806CAR antigen, EGFRvIII-His, followed by secondary staining using anti-His-PE. The level of staining was measured using FACS analysis (FIG. 5C). FIG. 5D depicts a bar graph of median fluorescence intensity (MFI) quantification of the PE dye in CD8+ cells. Selected cells were also stained for the EGFRt marker using Erbitux-biotin, and a secondary reagent, streptavidin-PE. The level of staining was measured using FACS analysis (FIG. 5E). FIG. 5F depicts a bar graph MFI quantification of the PE dye in CD8+ cells. Cell surface expression of 806CAR was significantly increased in cells containing the long-promoter-codon optimized construct (coHIV7.3) compared to cells containing the long promoter construct (HIV7.3) or short promoter construct (HIV7.2).

Expression and protein processing of the CARs in parallel to flow cytometry using western blot analysis (FIG. 6B). The density of each CAR molecule band on the western blot was quantified and normalized by the endogenous CD3 zeta signal, which was plotted as the bar graph (FIG. 6C). Gene copy number for the constructs in the cells was tested this using droplet digital (dd) PCR under standard methods (FIG. 6D, FIG. 6E). FIG. 6E depicts normalized CD3 zeta intensity from FIG. 6C and normalized again by the average copy number per cell. Cells containing the long promoter construct (HIV7.3) and human codon optimized construct (coHIV7.3) had a similar copy number, and this copy number was about half of that for cells containing the short promoter construct (HIV7.2). In view of the FACS analysis and Western blot analysis, the human codon optimized construct (coHIV7.3) had the expression efficiency of the three constructs. Constructs with the long promoter (EF1a(L)) had higher transcription activity than the construct with the short promoter (EF1a(S)). In addition, codon optimization improved gene expression.

### Example 3-Human Codon Optimized EGFR806CAR Construct Increased Cytokine Release

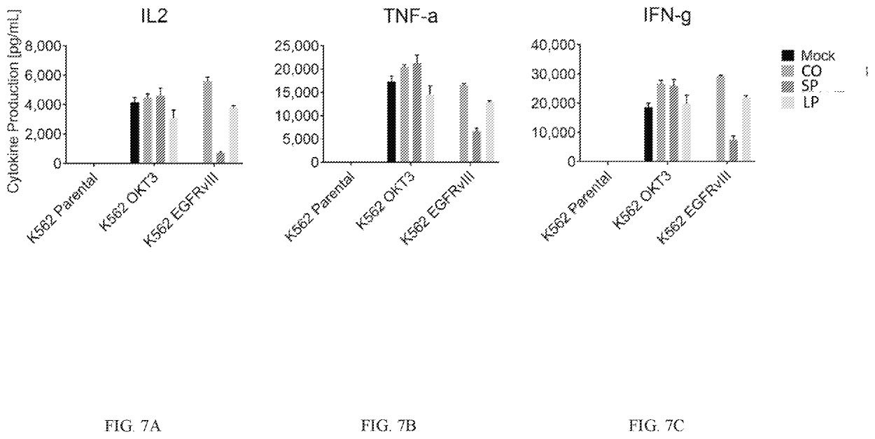

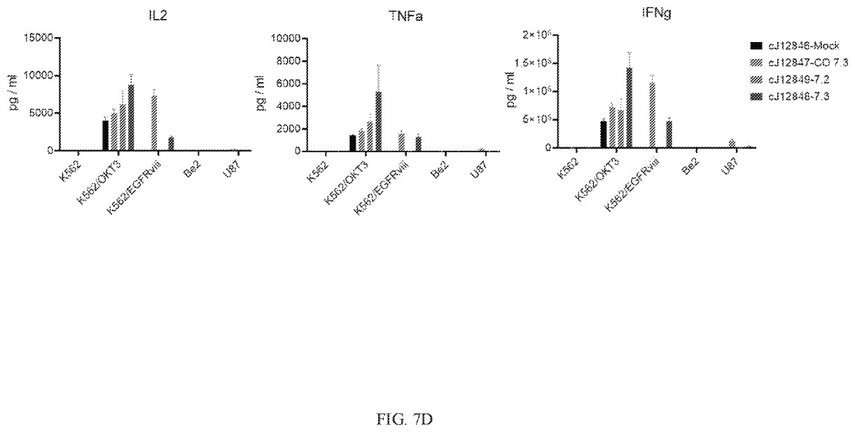

As disclosed herein, the inventors analyzed whether the enhanced expression from the human codon optimized EGFR806CAR construct led to enhanced cytokine release. This was tested using a cytokine release assay (BioPlex) (FIG. 7A, FIG. 7B, FIG. 7C). The K562 parental line and K562/OKT3 were used as the negative and positive controls, respectively. K563 is an antigen-presenting cell, and OKT3 is an anti-TCR antibody that is expressed on K562 as a positive control. K562/EGFRvIII line is a target line of 806CAR T cells. As depicted in the drawings, IL2, TNF-a, and IFN-g all showed enhanced release with the human codon optimized EGFR806CAR construct. This consistently demonstrated that higher EGFR806CAR expression correlates with greater cytokine release.

In additional experiments, human T cells from donors were transduced with constructs depicted in FIG. 1A, FIG. 1B, and FIG. 1C, and selected with methotrexate. The transduced effector cells were challenged with target cells expressing CAR targets in a cytokine release assay for IL2, TNFs and IFNg. K562 parental line and K562/OKT3 were negative and positive controls respectively. K562/EGFRvIII line was an 806CAR T cell target line. As shown in FIG. 7D, cells containing the coHIV7.3 construct had the highest cytokine release upon Ag+ tumor stimulation.

### Example 4-Human Codon Optimized EGFR806CAR Construct Functioned Optimally in Real Tumor Lines

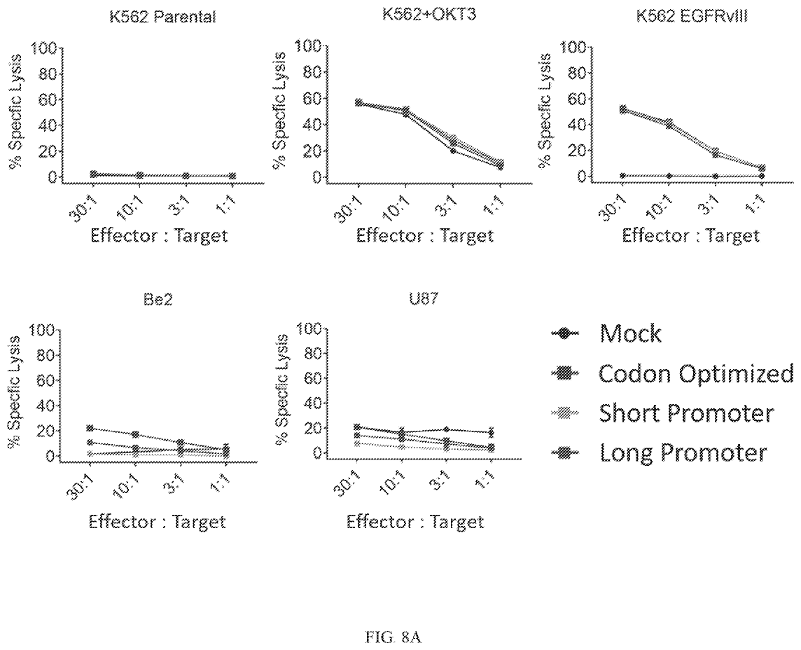

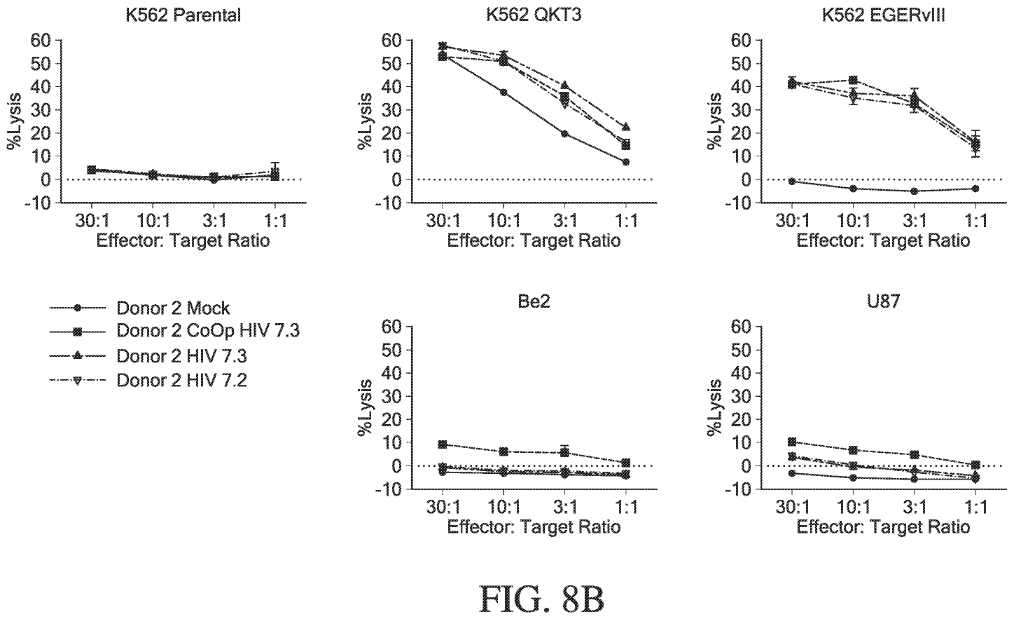

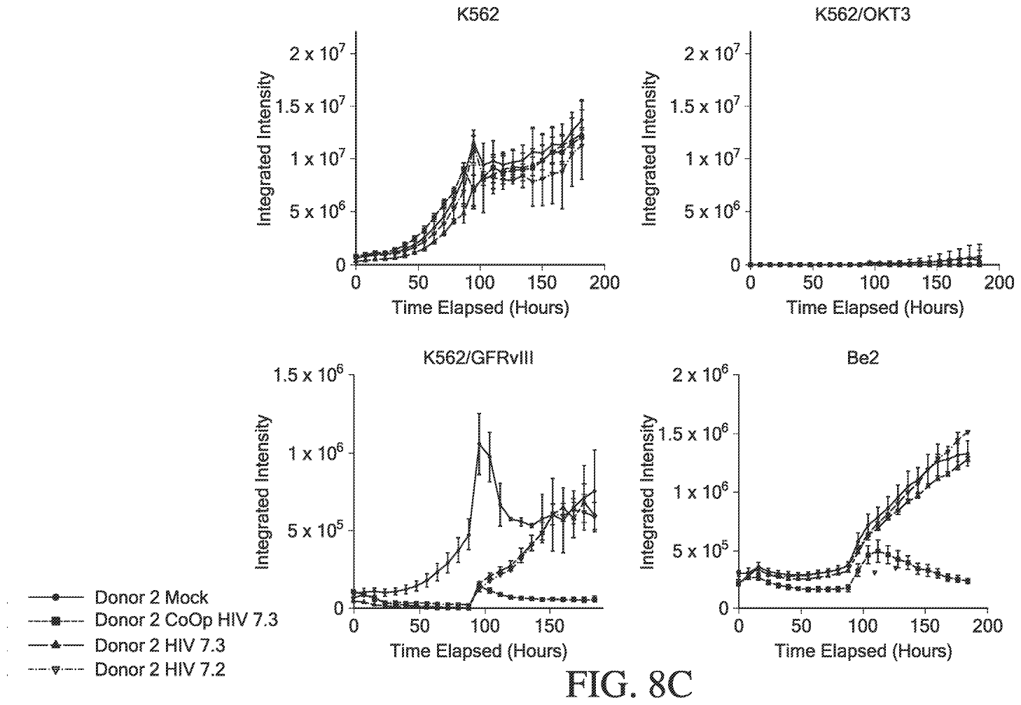

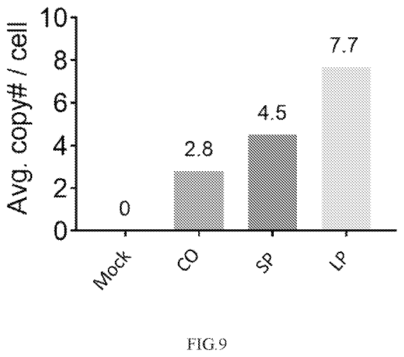

As disclosed herein, the inventors analyzed whether the enhanced expression from the human codon optimized EGFR806CAR construct correlated with cytotoxicity (FIG. 8A). Cytotoxicity was assessed using a chromium release assay under standard conditions. As in the cytokine assay, the K562 parental line and K562/OKT3 were used as negative and positive controls, respectively. K562/EGFRvIII line is an engineered target line of 806CAR T cells expressing exogenous EGFRvIII. The effector to target cell ratio ranged from 30:1 to 1:1. All three CAR T cells showed high cytotoxicity using K562 cells expressing EGFRvIII and differential cytotoxicity using Be2 and U87 tumor lines. The data disclosed herein provide evidence of the benefit of using codon optimized EGFR806CAR construct T cells for addressing low antigen expressing cells, which are usually real tumor lines, and not an artificial tumor line such as K562/EGFRvIII.

In additional experiments, human T cells from donors were transduced with constructs depicted in FIG. 1A. FIG. 1B, and FIG. 1C, and selected with methotrexate. The transduced effector cells were challenged with target cells expressing CAR targets in a cytotoxicity assay (chromium release assay). As shown in FIG. 8B, all 806CAR T cells showed effective killing of target Ag+ tumor cells. Cells containing coHIV7.2 had the highest cytolytic activity to the natural tumor lines, Be2 and U87 compared to cells containing HIV7.3 or HIV7.2.

In additional experiments, human T cells from donors were transduced with constructs depicted in FIG. 1A, FIG. 1B, and FIG. 1C, and selected with methotrexate. The transduced effector cells were challenged twice with different target tumor cells expressing mCherry marker at E:T ratio of 2:1 at 0 hr and 96 hr. The mCherry signal was collected by Incucyte. K562 cell line was a negative control, K562/OKTs was a positive control, K562/EGFRVIII and Be2 were CAR specific target lines. As shown in FIG. 8C, cells containing the coHIV7.3 construct significantly suppressed the growth ofK562/EGFRVIII and Be2 cells after each tumor challenge. Cells containing the HIV7.3 construct or the HIV7.2 construct could not suppress the K562/EGFRVII tumor cell growth after the second challenge. Cells containing the HIV7.3 construct or the HIV7.3 construct did not inhibit Be2 (a low tumor antigen cell line) tumor cell growth.

### Example 5-Human Codon Optimized EGFR806CAR Construct Conferred Significant Therapeutic Benefits In Vivo

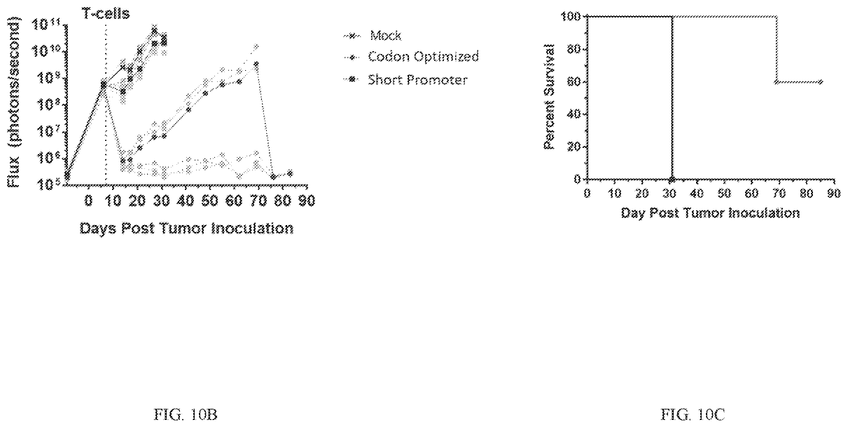

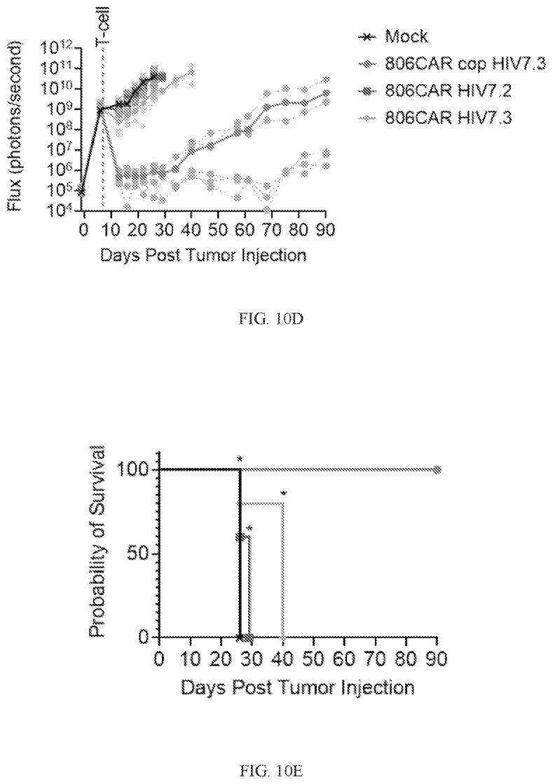

As disclosed herein, the performance of T cells containing the human codon optimized EGFR806CAR construct were tested in vim. Sequences containing the short promoter construct, or the long promoter and the human codon optimized construct were transduced into T cells before being studied in an intracranial NSG mouse model (FIG. 10A). T cells were used at a low dose (non-curative) to be able to detect the difference between the testing groups. Each testing group contained 5 mice. U87 glioma cells (806CAR target) expressing GFP:ffluc (GFP and firefly luciferase fusion protein) were injected intracranially (i.c.). A week later, T cells were i.c. injected. Bioluminescence images were taken at least once a week and the signal quantification is shown. As depicted in the drawings, mice with the human codon optimized EGFR806CAR construct had significantly lower bioluminescence after T cell injection, indicating a reduction in tumor formation (FIG. 10B). Similarly, these mice had a higher survival rate from tumor related death over-time compared with the negative control and mice given EGFR806CAR construct with a short promoter (FIG. 10C). Collectively, this data indicated that the human codon optimized EGFR806CAR construct with long promoter (SEQ ID NO: 10) has higher expression, enhanced cytokine release, greater performance in real tumor cells, and greater performance in vivo.

In additional experiments, human T cells from donors were transduced with constructs depicted in FIG. 1A. FIG. 1B, and FIG. 1C, and selected with methotrexate. Transduced cells were studied in the intracranial NSG mouse model. T cells were used at a low dose (non-curative) to be able to detect the difference between the testing groups. Each testing group contained 5 mice. U87 glioma cells (806CAR target) expressing GFP:fluc (GFP and firefly luciferase fusion protein) were injected intracranially (i.e.). A week later, T cells were i.c. injected. Bioluminescence images were taken at least once a week and the signal quantified (FIG. 10D). A Kaplan Meier analysis was performed (FIG. 10E). All mice injected with cells containing the coHIV7.3 construct survived through 90 days without symptoms. The rest of the mice were euthanized for tumor related symptoms.

1. A polynucleotide comprising a human codon-optimized sequence encoding an anti-EGFR chimeric antigen receptor (CAR).

2-58. (canceled)


SEQUENCE LISTING (10 sequences)

SEQ ID NO: 1 - DNA, 726 bp, Artificial Sequence
  Codon-optimized version of the sequence occuring in Homo Sapiens
gacgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgccggaagcacatctggctctggcaaacctggatctggcgagggctctaccaagggcgacatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggcggaggcacaaagctggaaatcaagcgc

SEQ ID NO: 2 - DNA, 544 bp, Homo sapiens
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 3 - DNA, 66 bp, Artificial Sequence
  Codon-optimized version of the sequence occuring in Porcine teschovirus-1 2A
ggaagcggcgccacaaatttcagcctgctgaaacaggccggcgacgtggaagagaaccctggacct

SEQ ID NO: 4 - DNA, 63 bp, Artificial Sequence
  Codon-optimized version of the sequence occuring in Thosea asigna virus 2A
ggcggaggcgaaggcagaggttctctgcttacatgcggagatgtggaagaaaatcccgggcct

SEQ ID NO: 5 - DNA, 561 bp, Artificial Sequence
  Codon-optimized version of the sequence occuring in Homo sapiens
atggtcggaagcctgaactgcatcgtggccgtgtctcagaacatgggcatcggcaagaacggcgacttcccttggcctcctctgagaaacgagagccggtacttccagcggatgaccaccacaagcagcgtggaaggcaagcagaacctggtcatcatgggcaagaaaacctggttcagcatccctgagaagaacagacccctgaagggcagaatcaacctggtgctgagcagagagctgaaagagcctcctcagggcgcccactttctgagcagatctctggacgatgccctgaagctgaccgagcaacctgagctggccaacaaggtggacatggtctggatcgttggcggcagcagcgtgtacaaagaagccatgaatcaccccggccacctgaaactgttcgtgaccagaatcatgcaggacttcgagagcgacacattcttcccagagatcgacctggaaaagtacaaactgctgcctgagtaccccggcgtgctgagcgacgtgcaagaagagaaaggcatcaagtacaagttcgaggtgtacgagaagaacgac

SEQ ID NO: 6 - DNA, 1005 bp, Artificial Sequence
  Codon-optimized version of the sequence occuring in Homo sapiens
cggaaagtgtgcaacggcatcggaatcggcgagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggatatcctgaaaaccgtgaaagagatcaccggatttctgttgatccaggcttggcccgagaaccggacagatctgcacgccttcgagaacctcgagatcatcagaggccggaccaagcagcacggccagttttctctggccgtggtgtccctgaatatcacctctctgggcctgcgcagcctgaaagaaatctccgatggcgacgtgatcatcagcggaaacaagaacctgtgctacgccaacaccatcaactggaagaagctgttcggcacctccggccagaaaacaaagatcatctccaaccggggcgagaactcctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctcgagggcgagccacgcgagttcgtggaaaacagcgagtgcatccagtgtcaccccgagtgcctgcctcaggccatgaacatcacatgcaccggaagaggccccgacaactgtatccagtgcgcccactatatcgacggccctcactgcgtgaaaacctgtcctgctggcgtgatgggagagaacaacaccctcgtgtggaagtacgccgatgccggacatgtgtgccacctgtgtcaccctaattgcacctacggctgtacaggcccaggactggaaggctgccctacaaacggacctaagatccccagcattgccaccggcatggttggagccctgctgcttctgctggtggtggcccttggaatcggactgtttatg

SEQ ID NO: 7 - DNA, 462 bp, Artificial Sequence
  Codon-optimized version of the sequence occuring in Homo sapiens
aagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccacaaaggacacctatgacgccctgcacatgcaggcccttccacctaga

SEQ ID NO: 8 - DNA, 84 bp, Artificial Sequence
  Codon-optimized version of the sequence occuring in Homo sapiens
atgttctgggtgctcgtggttgttggcggagtgctggcctgttatagcctgcttgtgaccgtggccttcatcatcttttgggtc

SEQ ID NO: 9 - DNA, 36 bp, Artificial Sequence
  Codon-optimized version of the sequence occuring in Homo sapiens
gagtctaagtacggccctccttgtcctccatgtcct

SEQ ID NO: 10 - DNA, 3698 bp, Artificial Sequence
  Sequence comprising the EFa1/HTLV promoter, as well as the sequence encoding a polypeptide comprising codon-optimized human EGFR806scFV, IgG4, CD28tm, 41BB, CD3?, P2A, DHFRdm, T2A, and EGFRt
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgttgctgctggttacatctctgctgctgtgcgagctgccccatcctgcctttctgctgatccctgacgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgccggaagcacatctggctctggcaaacctggatctggcgagggctctaccaagggcgacatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggcggaggcacaaagctggaaatcaagcgcgagtctaagtacggccctccttgtcctccatgtcctatgttctgggtgctcgtggttgttggcggagtgctggcctgttatagcctgcttgtgaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccacaaaggacacctatgacgccctgcacatgcaggcccttccacctagaggaagcggcgccacaaatttcagcctgctgaaacaggccggcgacgtggaagagaaccctggacctatggtcggaagcctgaactgcatcgtggccgtgtctcagaacatgggcatcggcaagaacggcgacttcccttggcctcctctgagaaacgagagccggtacttccagcggatgaccaccacaagcagcgtggaaggcaagcagaacctggtcatcatgggcaagaaaacctggttcagcatccctgagaagaacagacccctgaagggcagaatcaacctggtgctgagcagagagctgaaagagcctcctcagggcgcccactttctgagcagatctctggacgatgccctgaagctgaccgagcaacctgagctggccaacaaggtggacatggtctggatcgttggcggcagcagcgtgtacaaagaagccatgaatcaccccggccacctgaaactgttcgtgaccagaatcatgcaggacttcgagagcgacacattcttcccagagatcgacctggaaaagtacaaactgctgcctgagtaccccggcgtgctgagcgacgtgcaagaagagaaaggcatcaagtacaagttcgaggtgtacgagaagaacgacggcggaggcgaaggcagaggttctctgcttacatgcggagatgtggaagaaaatcccgggcctatgctgctgctcgtgacaagcctgctcctgtgtgaactccctcatccagcttttctgctcattccccggaaagtgtgcaacggcatcggaatcggcgagttcaaggacagcctgagcatcaacgccaccaacatcaagcacttcaagaactgcaccagcatcagcggcgacctgcacattctgcctgtggcctttagaggcgacagcttcacccacacacctccactggatccccaagagctggatatcctgaaaaccgtgaaagagatcaccggatttctgttgatccaggcttggcccgagaaccggacagatctgcacgccttcgagaacctcgagatcatcagaggccggaccaagcagcacggccagttttctctggccgtggtgtccctgaatatcacctctctgggcctgcgcagcctgaaagaaatctccgatggcgacgtgatcatcagcggaaacaagaacctgtgctacgccaacaccatcaactggaagaagctgttcggcacctccggccagaaaacaaagatcatctccaaccggggcgagaactcctgcaaggctacaggccaagtgtgccacgctctgtgtagccctgaaggctgttggggacccgagcctagagattgcgtgtcctgcagaaacgtgtcccggggcagagaatgcgtggacaagtgcaatctgctcgagggcgagccacgcgagttcgtggaaaacagcgagtgcatccagtgtcaccccgagtgcctgcctcaggccatgaacatcacatgcaccggaagaggccccgacaactgtatccagtgcgcccactatatcgacggccctcactgcgtgaaaacctgtcctgctggcgtgatgggagagaacaacaccctcgtgtggaagtacgccgatgccggacatgtgtgccacctgtgtcaccctaattgcacctacggctgtacaggcccaggactggaaggctgccctacaaacggacctaagatccccagcattgccaccggcatggttggagccctgctgcttctgctggtggtggcccttggaatcggactgtttatgtag